(19) United States
(12) Patent Application Publication
(10) Pub. No.: US 2008/0145340 A1
(43) Pub. Date: Jun. 19, 2008

(54) Neuroactive agents and methods of their use

(75) Inventors: Bruno Meloni, Mount Hawthorn (AU); Sherif Boulos, Dalheith (AU); Neville W. Kunckey, Double View (AU)

Correspondence Address: EDWARDS ANGELL PALMER & DODGE LLP, P.O. BOX 55874, BOSTON, MA, 02205, US

(73) Assignee: The University of Western Australia

(21) Appl. No.: 11/891,449

(22) Filed: Aug. 10, 2007

Related U.S. Application Data
(63) Continuation of application No. PCT/AU2006/000184, filed on Feb. 10, 2006.

(30) Foreign Application Priority Data
Feb. 10, 2005 (AU) 2005900614

(51) Int. Cl. A61K 38/52; C12N 5/00; C12N 15/85; A61K 48/00; A61P 25/00; A61P 25/28; G01N 33/567; C12Q 1/533

(52) U.S. Cl. 424/93.2; 435/375; 435/455; 435/7.21; 435/4; 424/94.5

(57) ABSTRACT

This invention is related to a method of controlling neurodegeneration by increasing CD 147 receptor signaling. Neuroprotection can be achieved suing cyclophilin A or a functional variant, analog or derivative as a ligand for the CDE 147 receptor administered in various means including gene therapy. Conditions treatable with this method include cerebra ischemia, Alzheimer's Disease, Parkinson's Disease, Motor Neurone Disease and/or N=neuronal loss due to trauma and spinal cord damage.

### RELATED APPLICATIONS

This is a continuation patent application that claims priority to PCT patent application number PCT/AU2006/000184, filed on Feb. 10, 2006, which claims priority to Australian patent application number 2005900614, filed on Feb. 10, 2005, the entirety of which are herein incorporated by reference.

### FIELD OF THE INVENTION

The present invention relates to the identification of a new target on neurons through which neuroprotection can be mediated. The present invention also relates to methods for controlling neurodegeneration by evoking or increasing CD147 receptor signalling on neurons. The present invention also relates to the use of agents adapted to bind CD147 such as cyclophilin A and functional variants thereof as neuroprotective agents. The invention also relates to methods of treatment and screening methods.

### BACKGROUND

Stroke research is based on the hypothesis that ischemia produces disability and death, not directly, but rather indirectly by initiating a cascade of cellular processes that eventually lead to neuronal death. As it is not presently feasible to regenerate functional neurons to replace dead ones, the best hope for an effective treatment for stroke is to intervene quickly with treatments that interrupt and reverse the cascade of events triggered by the primary ischemic event before they become irreversible.

Neuronal preconditioning occurs when a sublethal stress or stimuli induces neurons to become tolerant to a subsequent lethal ischemic insult. Preconditioning can induce acute and delayed tolerance. Acute preconditioning has a rapid onset, is not reliant on new protein synthesis, is mediated by post-translational protein activity and is short-lived. Delayed preconditioning, which has been more widely studied, is reliant on new protein synthesis, hence evolves after several hours and lasts for 1 to 7 days. Preconditioning treatments can induce neuronal ischemic tolerance in vivo, and in vitro using brain slices or dissociated neuronal cultures and is one of the most potent forms of neuroprotection against ischemic injury.

In terms of investigating the pathways involved in preconditioning, studies have either focused on early post-translational signaling events or late transcriptional (mRNA) and translational (protein) events. Despite evidence the neuroprotective preconditioning response is reliant on the expression of newly synthesised proteins, only a small number of proteins have been implicated (e.g. IL-1, Bcl-2, HSP70, EPO) and no study has endeavored to identify large scale protein changes. In addition, most studies have focused on ischemic preconditioning, despite the fact that other preconditioning treatments are likely to involve additional proteins targeting different events in neuronal death/survival pathways. Furthermore, no study has examined protein expression in a near-pure neuronal cell population, which would increase the probability of identifying protein changes important in ischemic tolerance specific to neurons.

The present invention seeks to overcome or at least partially alleviate the above problems by identifying a new target for neuroprotective agents.

### SUMMARY OF THE INVENTION

Applicants have identified a target for neuroprotective therapies and a novel neuroprotective agent. In particular, the applicants identified CyPA as a neuroprotective agent and have characterized its mode of action via CD147.

Thus, the present invention provides a method of controlling neurodegeneration by increasing CD147 receptor signalling on neurons.

The present invention also provides for the use of cyclophilin A (CyPA) or a functional variant thereof as a neuroprotectant.

The identification of the role of CD147 in the neuroprotection yields a new target for the development of other neuroprotectants. Thus, the present invention also provides a method for screening a compound for neuroactivity comprising contacting a candidate with CD147 and assessing binding and or receptor signalling.

The subject invention may also be used to screen patients for a predisposition to neurodegeneration. Thus the present invention also provides a screening method comprising the steps of: (i) detecting the presence and/or measuring the level at least one of CD147, CyPA or a functional variant thereof in a patient; and (ii) comparing the result from (i) with a reference measure indicative of normality.

The present invention also provides methods of treatment, pharmaceutical formulations.

### BRIEF DESCRIPTION OF THE FIGURES

FIG. 1 (A-D) is a table listing a number of proteins that were up or down regulated in neurons preconditioned with heat stress, cycloheximide or MK801;

FIG. 2 (A-D) is a table listing a number of proteins that were up or down regulated in neurons preconditioned with EPO;

FIG. 3A is a schematic of an expression cassette for recombinant adenovirus construction showing assembly of the transgene expression cassette containing the RSV promoter and WPRE, and the independent EGFP reporter cassette containing the CMV promoter. The viral vectors shown are the AdRSV:Empty (control virus) and the AdRSV:CyPA/WPRE, the shaded rectangle denotes SV40-polyadenylation signal sequence;

FIG. 3B—detection of CyPA mRNA expression by RT-PCR analysis of total RNA collected 72 hours following transfection of neuronal cultures with; AdRSV:Empty (moi of 100), AdRSV:Empty (moi of 100), AdRSV:CyPA/WPRE (moi of 500) and AdRSV:CyPA/WPRE (moi of 500). Endogenous expression of CyPA mRNA (indicated by the 465 bp PCR product) is evident in cultures transfected with AdRSV:Empty whilst AdRSV:CyPA/WPRE transfected cultures show both endogenous CyPA mRNA expression and viral mediated CyPA mRNA expression (indicated by the 535 bp PCR product);

FIG. 3C—western analysis of cortical neuronal cultures examined 72 hours after transfection with AdRSV:Empty (moi of 50) and AdRSV:CyPA/WPRE (moi of 50). Protein lysates were probed with anti-CyPA antibody and show increased CyPA expression in neuronal cultures transfected with AdRSV:CyPA/WPRE;

FIG. 3D Immunohistochemical staining of cortical neuronal cultures transfected on DIV 9 with AdRSV:Empty (moi of 100) and AdRSV:CyPA/WPRE (moi of 100) and examined 72 hours later. Cultures were probed with anti-CyPA antibody and stained with DAB, and show increased CyPA expression in neuronal cultures transfected with AdRSV:CyPA/WPRE;

FIG. 3E—immunofluorescence of cortical neuronal cultures showing localisation of CyPA expression in neurons a) control cultures probed with rabbit anti-CyPA antibody and goat anti-mouse IgG AlexaFluor 488 secondary antibody, showing absence of non-specific CyPA immunoreactivity; b) control cultures probed with rabbit anti-CyPA antibody and goat anti-rabbit IgG AlexaFluor 546 secondary antibody showing CyPA immunoreactivity; c) control cultures probed with mouse monoclonal anti-GFAP (1:500; Sigma) and goat anti-mouse IgG AlexaFluor 488 secondary antibody showing GFAP immunoreactivity (d) control cultures probed with mouse monoclonal anti-GFAP (1:500; Sigma) and goat anti-mouse IgG AlexaFluor 488 control to indicate extent of non specific fluorescence; e) probed with rabbit anti-CyPA and mouse monoclonal anti-GFAP (1:500; Sigma) and detected with goat anti-rabbit IgG AlexaFluor 546 and goat anti-mouse IgG AlexaFluor 488 (Molecular Probes);

FIG. 4—transfection of neuronal cultures with AdRSV:CyPA/WPRE and the control vector AdRSV:Empty. (A) Cortical neuronal cultures were transfected with recombinant adenovirus (moi of 75) on DIV 9. At 72 hours post-transfection, cultures were exposed to either cumene hydroperoxide (25 μM), with or without glutamate blockers or sham treated. Neuronal survival was assessed 24 hours later (n=4, *P<0.05). (B) Cortical neuronal cultures were transfected with recombinant adenovirus (moi of 75) on DIV 9. At 72 hours post-transfection, cultures were either exposed to in vitro ischemia, with or without glutamate blockers or sham treated. Neuronal survival was assessed 24 hours later (n=4, *P<0.05);

FIG. 5—In vivo detection of CyPA mRNA and protein following global cerebral ischemia in the rat hippocampus. (A) Time course of CyPA mRNA following 3 mins of preconditioning ischemia, showing a significant increase at 24 hours post-ischemia, but not at 6 hours post ischemia. (B) Western analysis of total protein probed with anti-CyPA antibody and showing no difference in CyPA levels between a sham treatment group (a, b, c; n=3) and those treated with 3 mins of preconditioning ischemia (d, e, f; n=3);

FIG. 6—Immunodetection of CD147 receptor protein (A) Western blot of total protein from rat hippocampus (HP) and rat cortical neuronal cultures (CNC) probed with anti-CD147 antibody showing immunoreactive protein. (B) Photomicrographs of cortical neuronal cultures showing immunocytochemical staining of CD147. Cultures were probed with: no antibody (control); anti-glial fibrillary acidic protein (GFAP) antibody; anti-neuron specific enolase (NSE) antibody; or anti-CD147 antibody, stained with DAB. Cultures probed with NSE antibody and anti-CD147 display a similar pattern. (C) Photomicrographs of astrocyte enriched neuronal cultures showing immunocytochemical staining of CD147. Cultures were probed with either; no antibody (control); anti glial fibrillary acidic protein (GFAP) antibody; anti-neuron specific enolase (NSE) antibody; or anti-CD147 antibody (panel D), and stained with DAB. Cultures probed with NSE antibody and anti-CD147 display a similar pattern;

FIG. 7—time course of recombinant human (rh) CyPA mediated ERK1/2 phosphorylation in cortical neuronal cultures. Neuronal cultures were treated with rhCyPA (100 nM) for the indicated times. The resulting protein lysates were probed with antibody to detect phosphorylated ERK1/2 and then re-probed for total ERK1/2. Graph shows results of quantitation of the immunoblots using densitometry showing relative ERK1/2 activation for each time point; and

FIG. 8—effect of exogenous application of rhCyPA on neuronal survival following oxidative stress and in vitro ischemia. (A) Cortical neuronal cultures were exposed to cumene hydroperoxide (25 μM), and treated with rhCyPA at the indicated doses or with glutamate blockers. Neuronal survival was assessed 24 hours later (n=4, *P<0.05). (B) Cortical neuronal cultures were exposed to in vitro ischemia, and treated with rhCyPA at the indicated doses or with glutamate blockers. Neuronal survival was assessed 24 hours later (n=4, *P<0.05).

### DETAILED DESCRIPTION OF THE INVENTION

### Methods of Controlling Neurodegeneration

The present invention provides a method of controlling neurodegeneration by increasing CD147 receptor signalling on neurons.

CD147 receptor signalling can be increased through the use of a ligand adapted to bind CD147 and evoke receptor signalling.

Alternatively, CD147 receptor signalling can be increased by increasing the expression of CD147 on neurons or increasing signalling efficiency.

CD147 expression on neurons can be increased in a variety of ways. Preferably, the expression is increased via DNA based therapies that are described in more detail hereunder. Essentially, DNA encoding CD147 is introduced into neurons to result in an increase in CD147 expression relative to non-treated cells. The introduced DNA could be adapted to be transcribed at high levels. Additionally or alternatively, the introduced DNA could encode a modified CD147 that has enhanced ligand binding affinity or some other characteristic that renders it capable of increased CD147 receptor signalling.

CD147 expression could also be increased through the use of an agent that (i) increases transcription of the CD147 DNA into mRNA and/or (ii) increases the translation of mRNA coding for CD147.

### Use of Cyclophilin a and Functional Variants Thereof as a Neuroprotectant

The present invention provides for the use of cyclophilin A (CyPA) or a functional variant thereof as a neuroprotectant.

Whilst the applicant does not wish to be bound by any particular mode of action there is evidence that CyPA exerts its neuroprotective activity via CD147 receptor signalling and/or activation of the ERK1/2 pro-survival pathways.

A “functional variant” for the purposes of the present invention include peptides and non-peptide mimetics that retain at least one important characteristic of CyPA such as its neuroprotective activity and/or its ability to evoke CD147 receptor signalling. Cyclophilin B and C are two examples of peptides that comprise functional variants of the present invention. The peptides may be recombinant, natural or synthetic. Preferably, the polypeptides are recombinant. Methods for screening for functional variants including agonists are described in more detail hereunder.

Thus, functional variants of the invention also include variants of CyPA with deletions, insertions, inversions, repeats, and type substitutions. Guidance concerning which amino acid changes are likely to be phenotypically silent can be found in Bowie, J. U., et al, “Deciphering the Message in Protein Sequences: Tolerance to Amino Acid Substitutions,” Science 247:1306-1310 (1990).

A functional variant of CyPA may be: (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which CyPA is fused with another compound, such as a compound to increase the half life of CyPA (for example, polyethylene glycol or polypropylene glycol), or (iv) one in which the additional amino acids are fused to CyPA, such as a leader or secretory sequence or a sequence which is employed for purification or a proprotein sequence. Such fragments, derivatives and analogs are deemed to be within the scope of term functional variants for the purposes of the present invention.

Of particular interest are the replacement of amino acids that alter the neuroactivity or binding affinity of CyPA. Thus, the functional variants of the present invention may include one or more amino acid substitutions, deletions or additions, relative to native CyPA, either from natural mutations or human manipulation. The particular replacements may be determined by a skilled person as detailed more fully hereunder. However, changes are preferably of a minor nature, such as conservative amino acid substitutions that do not significantly affect the folding or activity of the protein (see for example the table hereunder). Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Amino acids in CyPA that are essential for function, such as neuroprotectivity and/or CD147 receptor binding, can be identified by methods known in the art, such as site directed mutagenesis or alanine-scanning mutagenesis. The latter procedure introduces single alanine mutations at every residue in the molecule. The resulting mutant molecules are then tested for biological activity such as neuroactivity or ability to evoke CD147 receptor signalling. Sites that are critical for ligand-receptor binding can also be determined by structural analysis such as crystallization. Nuclear magnetic resonance or photoaffinity labelling may also be used when developing functional variants. Alternatively, synthetic peptides corresponding to candidate functional variants may be produced and their ability to display neuroactive properties in vitro or in vivo.

Functional variants of CyPA can be prepared as libraries having sequences based on the sequence of CyPA, but with various changes. Phage display can also be effective in identifying functional variants with useful neuroprotective properties.

Briefly, one prepares a phage library (using e.g. ml3, fd, or lambda phage), displaying inserts from 4 to about 80 amino acid residues using conventional procedures. The inserts may represent, for example, a biased degenerate array or may completely restrict the amino acids at one or more positions within CyPA. One can then select phage-bearing inserts that have a relevant biological activity of CyPA such as neuroactivity or receptor binding/signalling. This process can be repeated through several cycles of reselection of phage. Repeated rounds lead to enrichment of phage bearing particular sequences. DNA sequence analysis can be conducted to identify the sequences of the expressed polypeptides. The minimal linear portion of the CyPA sequence that confers the relevant activity can be determined. One can repeat the procedure using a biased library containing inserts containing part or the entire minimal linear portion plus one or more additional degenerate residues upstream or downstream thereof.

Functional variants of CyPA can be tested for retention of any of the useful properties of CyPA. For example, they can be tested for in vitro properties, initially on neuronal cells, to determine which ones retain neuroactivity. One in vitro property indicative of a useful neuroprotective agent is the ability of a functional variant to prolong the survival of neurons in culture. Peptides that retain or lack a relevant property can then be used in in vivo assays of neuroprotection such as the in vivo and in vitro assays described in the Examples section herein.

Preferred functional variants of the present invention comprise an amino acid sequence that is at least 70-80% identical, more preferably at least 90% or 95% identical, still more preferably at least 96%, 97%, 98% or 99% identical to CyPA.

By a polypeptide having an amino acid sequence at least, for example, 95% “identical” to a reference amino acid sequence it is intended that the amino acid sequence of the polypeptide is identical to the reference sequence except that the polypeptide sequence may include up to five amino acid alterations per each 100 amino acids of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to CyPA can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters are set, of course, such that the percentage of identity is calculated over the full length of the reference amino acid sequence and that gaps in homology of up to 5% of the total number of amino acid residues in the reference sequence are allowed.

In general, the functional variants of the present invention can be synthesized directly or obtained by chemical or mechanical disruption of larger molecules, fractioned and then tested for one or more activity of the native molecule such as neuroactivity. Functional variants with useful properties may also be obtained by mutagenesis of a specific region of the nucleotide encoding the polypeptide, followed by expression and testing of the expression product, such as by subjecting the expression product to in vitro tests on neuronal cells to assess its neuroactivity and/or receptor binding. Functional variants may also be produced by Northern blot analysis of total cellular RNA followed by cloning and sequencing of identified bands derived from different tissues/cells, or by PCR analysis of such RNA also followed by cloning and sequencing. Thus, synthesis or purification of an extremely large number of functional variants is possible using the information contained herein.

Functional variants also include conformationally constrained peptides. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure.

The active conformation of a peptide may be stabilized by a covalent modification, such as cyclization or by incorporation of gamma-lactam or other types of bridges. For example, side chains can be cyclized to the backbone to create an L-gamma-lactam moiety on each side of the interaction site. Cyclization also can be achieved, for example, by formation of cysteine bridges, coupling of amino and carboxy terminal groups of respective terminal amino acids, or coupling of the amino group of a Lys residue or a related homolog with a carboxy group of Asp, Glu or a related homolog. Coupling of the alpha-amino group of a polypeptide with the epsilon-amino group of a lysine residue, using iodoacetic anhydride, can be also undertaken.

Another approach is to include a metal-ion complexing backbone in the peptide structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the peptide chain includes nitrogen atoms with amine, amide, imidazole, or guanidino functionalities; sulphur atoms of thiols or disulfides; and oxygen atoms of hydroxy, phenolic, carbonyl, or carboxyl functionalities. In addition, the peptide chain or individual amino acids can be chemically altered to include a coordinating group, such as for example oxime, hydrazino, sulfhydryl, phosphate, cyano, pyridino, piperidino, or morpholino. The peptide construct can be either linear or cyclic. However a linear construct is typically preferred. One example of a small linear peptide is Gly-Gly-Gly-Gly that has four nitrogens (an N₄ complexation system) in the backbone that can complex to a metal ion with a coordination number of four.

Functional variants of the present invention may also be determined by relying upon the development of amino acid sequence motifs to which potential epitopes may be compared. Each motif describes a finite set of amino acid sequences in which the residues at each (relative) position may be (a) restricted to a single residue, (b) allowed to vary amongst a restricted set of residues, or (c) allowed to vary amongst all possible residues. For example, a motif might specify that the residue at a first position may be any one of valine, leucine, isoleucine, methionine, or phenylalanine; that the residue at the second position must be histidine; that the residue at the third position may be any amino acid residue; that the residue at the fourth position may be any one of the residues valine, leucine, isoleucine, methionine, phenylalanine, tyrosine or tryptophan; that the residue at the fifth position must be lysine, and so on.

Sequence motifs for CyPA can be developed further by analysis of its structure and conformation. By providing a detailed structural analysis of the residues involved in forming the contact surfaces of the peptide, one is enabled to make predictions of sequence motifs that have similar binding properties.

Using these sequence motifs as search, evaluation, or design criteria, one is enabled to identify classes of peptides, that represent functional variants of CyPA, that have a reasonable likelihood of binding to the target and inducing a desired biological effect. These peptides can be synthesized and tested for activity as described herein. Use of these motifs, as opposed to pure sequence homology or sequence homology with unlimited “conservative” substitutions, represents a method by which one of ordinary skill in the art can further evaluate peptides for potential application in the treatment of the neurodegenerative effects of cerebrovascular ischemia, stroke and the like.

Thus, the present invention also provides methods for identifying functional variants of CyPA. In general, a first amino acid residue of CyPA is mutated to prepare a variant peptide. In one embodiment, the amino acid residue can be selected and mutated as indicated by a computer model of peptide conformation. Peptides bearing mutated residues that maintain a similar conformation (e.g. secondary structure) can be considered potential functional variants that can be tested for function using the assays described herein. Any method for preparing variant peptides can be employed, such as synthesis of the variant peptide, recombinantly producing the variant peptide using a mutated nucleic acid molecule, and the like. The properties of the variant peptide in relation to CyPA are then determined according to standard procedures as described herein.

Functional variants prepared by any of the foregoing methods can be sequenced, if necessary, to determine the amino acid sequence and thus deduce the nucleotide sequence which encodes such variants.

The functional variants of CyPA also extend to CyPA fragments. Preferably, the fragments retain neuroactivity, such as neuroprotection, or may be made intentionally to reduce or remove a biological activity of the polypeptide.

Other polypeptides fragments of the present invention are those that comprise the amino acid sequence of CyPA and lack a continuous series of residues (that is, a continuous region, part or portion) that includes the amino terminus, or a continuous series of residues that includes the carboxyl terminus or, as in double truncation mutants, deletion of two continuous series of residues, one including the amino terminus and one including the carboxyl terminus. Again, these truncation mutants preferably retain at least one biological activity of the full polypeptide such as their neuroactivity or their ability to bind to their receptor.

Preferably, the fragments of CyPA comprise at least 10, 20, 30, 50 or 100 amino acid residues. Preferably, the fragments include at least one biological activity of the full CyPA, such as neuroactivity and/or ability to bind a receptor for the full molecule or an antibody thereto.

Fragments or portions of the polypeptides of the present invention may be employed for producing the corresponding full-length polypeptide by peptide synthesis; therefore, the fragments may be employed as intermediates for producing the full-length polypeptides.

Representative examples of polypeptide fragments of the invention include those which are about 5-15, 10-20, 15-40, 30-55, 41-75, 41-80, 41-90, 50-100, 75-100, 90-115, 100-125 and 110-130 amino acids in length. In this context “about” includes the particularly recited range and ranges larger or smaller by several, a few, 5, 4, 3, 2 or 1 amino acid residues at either extreme or at both extremes. For instance, about 40-90 amino acids in this context means a polypeptide fragment of 40 plus or minus several, a few, 5, 4, 3, 2 or 1 amino acid residues to 90 plus or minus several a few, 5, 4, 3, 2 or 1 amino acid residues, i.e., ranges as broad as 40 minus several amino acids to 90 plus several amino acids to as narrow as 40 plus several amino acids to 90 minus several amino acids. Highly preferred in this regard are the recited ranges plus or minus as many as 5 amino acids at either or at both extremes. Particularly highly preferred are the recited ranges plus or minus as many as 3 amino acids at either or at both the recited extremes. Especially particularly highly preferred are ranges plus or minus 1 amino acid at either or at both extremes of the recited ranges with no additions or deletions. Most highly preferred of all in this regard are fragments from 5-15, 10-20, 15-40, 30-55, 41-75, 41-80, 41-90, 50-100, 75-100, 90-115, 100-125, and 110-130 amino acids long.

Other fragments of the present invention comprise an epitope-bearing portion of CyPA. Preferably, the epitope is an immunogenic or antigenic epitope of the polypeptide. An “immunogenic epitope” is defined as a part of a protein that elicits an antibody response when the whole protein is the immunogen. On the other hand, a region of a protein molecule to which an antibody can bind is defined as an “antigenic epitope.”

As to the selection of fragments bearing an antigenic epitope (i.e., that contain a region of a protein to which an antibody can bind), it is well known in that art that relatively short synthetic peptides that mimic part of a protein sequence are routinely capable of eliciting an antiserum that reacts with the partially mimicked protein. Peptides capable of eliciting protein-reactive sera are frequently represented in the primary sequence Z-1 of a protein, can be characterized by a set of simple chemical rules, and are confined neither to immunodominant regions of intact proteins (i.e. immunogenic epitopes) nor to the amino or carboxyl terminals. Antigenic epitope-bearing fragments of the invention are therefore useful to raise antibodies, including monoclonal antibodies that bind specifically to CyPA.

Antigenic epitope-bearing fragments of CyPA preferably contain a sequence of at least 7, 9 or at least about 15 to about 30 amino acids contained within the amino acid sequence of CyPA and may be contiguous or conformational epitopes. The epitope-bearing fragments the invention may be produced by any conventional means apparent to those skilled in the art.

Functional variants for the purposes of the present invention also include mimetics. Nonpeptide analogs of CyPA peptides, e.g., those that provide a stabilized structure or lessened biodegradation, are contemplated. Peptide mimetic analogs can be prepared based on a selected peptide by replacement of one or more residues by nonpeptide moieties. Preferably, the nonpeptide moieties permit the peptide to retain its natural conformation, or stabilize a preferred, e.g., bioactive, conformation.

A wide variety of useful techniques may be used to elucidating the precise structure of a peptide. These techniques include amino acid sequencing, x-ray crystallography, mass spectroscopy, nuclear magnetic resonance spectroscopy, computer-assisted molecular modelling, peptide mapping, and combinations thereof. Structural analysis of a peptide generally provides a large body of data that comprise the amino acid sequence of the peptide as well as the three-dimensional positioning of its atomic components. From this information, non-peptide peptidomimetics may be designed that have the required chemical functionalities for therapeutic activity but are more stable, for example less susceptible to biological degradation.

CyPA and functional variants thereof may also be provided conjugated to another molecule that confers another advantageous property. Fusion proteins, where another peptide sequence is fused to CyPA to aid in extraction and purification is one example. Examples of fusion protein partners include glutathione-S-transferase (GST), hexahistidine, GAL4 (DNA binding and/or transcriptional activation domains) and β-galactosidase. It may also be convenient to include a proteolytic cleavage site between the fusion protein partner and CyPA or functional variants thereof to allow removal of fusion protein sequences. Preferably, the fusion protein will not hinder an important activity of the protein such as neuroactivity and/or receptor binding. Fusion proteins including a peptide adapted to target CyPA or the function equivalent to a cell type or tissue is another example.

CyPA and functional variants thereof can also be conjugated to a moiety such as a fluorescent, radioactive, or enzymatic label (e.g. a detectable moiety, such as green fluorescent protein) or a molecule that enhances the stability of CyPA or the functional variant under assay conditions.

Preferably, the CyPA or functional variant thereof is conjugated to a compound that facilitates its transport across the blood-brain barrier (BBB). As used herein, a compound which facilitates transport across the BBB is one which, when conjugated to CyPA or a functional variant thereof, facilitates the amount of peptide delivered to the brain as compared with non-conjugated peptide. The compound can induce transport across the BBB by any mechanism, including receptor-mediated transport, and diffusion.

Compounds which facilitate transport across the BBB include transferrin receptor binding antibodies; certain lipoidal forms of dihydropyridine; carrier peptides, such as cationized albumin or Met-enkephalin; cationized antibodies; fatty acids such as docosahexaenoic acid (DHA) and C8 to C24 fatty acids with 0 to 6 double bonds, glyceryl lipids, cholesterol, polyarginine (e.g., RR, RRR, RRRR) and polylysine (e.g., KK, KKK, KKKK). Unbranched, naturally occurring fatty acids embraced by the invention include C8:0 (caprylic acid), C10:0 (capric acid), C12:0 (lauric acid), C14:0 (myristic acid), C16:0 (palmitic acid), C16:1 (palmitoleic acid), C16:2, C18:0 (stearic acid), C18:1 (oleic acid), C18:1-7 (vaccenic), C18:2-6 (linoleic acid), C18:3-3 (.alpha.-linolenic acid), C18:3-5 (eleostearic), C18:3-6 (&-linolenic acid), C18:4-3, C20:1 (gondoic acid), C20:2-6, C20:3-6 (dihomo-y-linolenic acid), C20:4-3, C20:4-6 (arachidonic acid), C20:5-3 (eicosapentaenoic acid), C22:1 (docosenoic acid), C22:4-6 (docosatetraenoic acid), C22:5-6 (docosapentaenoic acid), C22:5-3 (docosapentaenoic), C22:6-3 (docosahexaenoic acid) and C24: 1-9 (nervonic). Highly preferred unbranched, naturally occurring fatty acids are those with between 14 and 22 carbon atoms. The most preferred fatty acid is docosahexaenoic acid. Other BBB carrier molecules and methods for conjugating such carriers to peptides will be known to one of ordinary skill in the art. Such BBB transport molecules can be conjugated to one or more ends of the peptide.

CyPA can be conjugated to such compounds by well-known methods, including bifunctional linkers, formation of a fusion polypeptide, and formation of biotin/streptavidin or biotin/avidin complexes by attaching either biotin or streptavidin/avidin to the peptide and the complementary molecule to the BBB-transport facilitating compound. Depending upon the nature of the reactive groups in an isolated peptide and a targeting agent or blood-brain barrier transport compound, a conjugate can be formed by simultaneously or sequentially allowing the functional groups of the above-described components to react with one another. For example, the transport-mediating compound can be prepared with a sulfhydryl group at, e.g., the carboxyl terminus, which then is coupled to a derivatizing agent to form a carrier molecule. Next, the carrier molecule is attached via its sulfhydryl group, to the peptide. Many other possible linkages are known to those of skill in the art.

Conjugates of CyPA and a targeting agent or BBB transport-facilitating compound are formed by allowing the functional groups of the agent or compound and the peptide to form a linkage, preferably covalent, using coupling chemistries known to those of ordinary skill in the art. Numerous art-recognized methods for forming a covalent linkage can be used. See, for example, March, J., Advanced Organic Chemistry, 4th Ed., New York, N.Y., Wiley and Sons, 1985), pp. 326-1 120.

In the event that CyPA exhibits reduced activity in a conjugated form, the covalent bond between the CyPA and the BBB transport-mediating compound can be selected to be sufficiently labile (e.g., to enzymatic cleavage by an enzyme present in the brain) so that it is cleaved following transport of the peptides across the BBB, thereby releasing the free peptide to the brain. Biologically labile covalent linkages, e.g., imino bonds, and “active” esters can be used to form prodrugs where the covalently coupled peptides is found to exhibit reduced activity in comparison to the activity of the peptides alone.

It is envisioned that CyPA and functional variants described herein can be delivered to neuronal cells by site-specific means. Cell-type-specific delivery can be provided by conjugating a peptide to a targeting molecule, e.g., one that selectively binds to a target neuronal cell. One example of a well-known targeting vehicle is liposomes. Liposomes are commercially available from Gibco BRL (Gaithersburg, Md.). Numerous methods are published for making targeted liposomes. Liposome delivery can be provided by encapsulating an isolated polypeptide of the present invention in liposomes that include a cell-type-specific targeting molecule. Methods for targeted delivery of compounds to particular cell types are well-known to those of skill in the art.

In the absence of a free amino- or carboxyl-terminal functional group that can participate in a coupling reaction, such a group can be introduced, e.g., by introducing a cysteine (containing a reactive thiol group) into the peptide by synthesis or site directed mutagenesis. Disulfide linkages can be formed between thiol groups in, for example, the peptide and the BBB transport-mediating compound. Alternatively, covalent linkages can be formed using bifunctional crosslinking agents, such as bismaleimidohexane (which contains thiol-reactive maleimide groups and which forms covalent bonds with free thiols). See also the Pierce Co. Immunotechnology Catalogue and Handbook Vol. 1 for a list of exemplary homo- and hetero-bifunctional crosslinking agents, thiol-containing amines and other molecules with reactive groups.

In general, the conjugated peptides of the invention can be prepared by using well-known methods for forming amide, ester or imino bonds between acid, aldehyde, hydroxy, amino, or hydrazo groups on the respective conjugated peptide components. As would be apparent to one of ordinary skill in the art, reactive functional groups that are present in the amino acid side chains of the peptide (and possibly in the BBB transport-mediating compound) preferably are protected, to minimize unwanted side reactions prior to coupling the peptide to the derivatizing agent and/or to the extracellular agent. As used herein, “protecting group” refers to a molecule which is bound to a functional group and which may be selectively removed therefrom to expose the functional group in a reactive form. Preferably, the protecting groups are reversibly attached to the functional groups and can be removed therefrom using, for example, chemical or other cleavage methods. Thus, for example, the peptides of the invention can be synthesized using commercially available side-chain-blocked amino acids (e.g., FMOC-derivatised amino acids from Advanced Chemtech Inc., Louisville, Ky.). Alternatively, the peptide side chains can be reacted with protecting groups after peptide synthesis, but prior to the covalent coupling reaction. In this manner, conjugated peptides of the invention can be prepared in which the amino acid side chains do not participate to any significant extent in the coupling reaction of the peptide to the BBB transport-mediating compound or cell-type-specific targeting agent.

It will be appreciated that the amino acids in the peptides of the present invention that are required for neuroactivity and/or receptor binding may be incorporated into larger peptides and still maintain their function. Preferably, the amino acids required for neuroactivity are a contiguous sequence of between about 5 and 20 amino acids and more preferably between about 6 and 15 amino acids.

Preferably, the CyPA or functional variant thereof are non-hydrolyzable in that the bonds linking the amino acids of the peptide are less readily hydrolyzed than peptide bonds formed between L-amino acids. To provide such peptides, one may select isolated peptides from a library of non-hydrolyzable peptides, such as peptides containing one or more D-amino acids or peptides containing one or more non-hydrolyzable peptide bonds linking amino acids.

Alternatively, one can select peptides that are optimal for a preferred function (e.g. neuroprotective effects) in assay systems described in the Examples and then modify such peptides as necessary to reduce the potential for hydrolysis by proteases. For example, to determine the susceptibility to proteolytic cleavage, peptides may be labelled and incubated with cell extracts or purified proteases and then isolated to determine which peptide bonds are susceptible to proteolysis, e.g., by sequencing peptides and proteolytic fragments. Alternatively, potentially susceptible peptide bonds can be identified by comparing the amino acid sequence of an isolated peptide with the known cleavage site specificity of a panel of proteases. Based on the results of such assays, individual peptide bonds that are susceptible to proteolysis can be replaced with non-hydrolyzable peptide bonds by in vitro synthesis of the peptide.

Many non-hydrolyzable peptide bonds are known in the art, along with procedures for synthesis of peptides containing such bonds. Non-hydrolyzable bonds include -psi[CH.sub.2 NH]— reduced amide peptide bonds, -psi[COCH.sub.2]— ketomethylene peptide bonds, -psi[CH(CN)NH]— (cyanomethylene)amino peptide bonds, -psi[CH.sub.2 CH(OH)]— hydroxyethylene peptide bonds, -psi[CH.sub.2 O]— peptide bonds, and -psi[CH.sub.2 S]— thiomethylene peptide bonds.

Likewise, various changes may be made including the addition of various side groups that do not affect the manner in which the peptide functions, or which favourably affect the manner in which the peptide functions. Such changes may involve adding or subtracting charge groups, substituting amino acids, adding lipophilic moieties that do not affect binding but that affect the overall charge characteristics of the molecule facilitating delivery across the blood-brain barrier, etc. For each such change, no more than routine experimentation is required to test whether the molecule functions according to the invention. One simply makes the desired change or selects the desired peptide and applies it in a fashion as described in detail in the examples.

One approach is to link the CyPA or functional variant thereof to a variety of polymers, such as polyethylene glycol (PEG) and polypropylene glycol (PPG). Replacement of naturally occurring amino acids with a variety of uncoded or modified amino acids such as D-amino acids and N-methyl amino acids may also be used to modify peptides. Another approach is to use bifunctional crosslinkers, such as N-succinimidyl 3-(2 pyridyldithio)propionate, succinimidyl 6-[3-(2 pyridyldithio)propionamido]hexanoate, and sulfosuccinimidyl 6-[3-(2 pyridyldithio)propionamido]hexanoate.

### Screening Methods, Agonists and Antagonists

The present invention also provides agonists, antagonists and methods of screening compounds to identify those that enhance or block the binding of CyPA or functional variants thereof.

For example, a preparation containing neuronal cells or isolated receptors for CyPA, such as CD147, may be contacted with labelled CyPA in the absence or the presence of a candidate molecule that may be an agonist or antagonist. The ability of the candidate molecule to bind the receptor itself is reflected in decreased binding of the labelled CyPA. Molecules that bind gratuitously, i.e., without conferring neuroprotection, are most likely to be good antagonists. Molecules that bind and confer neuroprotection are likely to be good agonists.

The effects of potential agonists and antagonists on neurons may by measured, for instance, by exposing neurons to ischemia concurrently or prior to dosing with the antagonist or agonist, and comparing the effect with suitable controls.

Another example of an assay for antagonists is a competitive assay that combines CyPA and a potential antagonist with neurons or receptors therefrom such as CD147 under appropriate conditions for a competitive inhibition assay. The CyPA can be labelled, such as by radioactivity, such that its binding to the neuron or receptor can be determined accurately to assess the effectiveness of the potential antagonist.

Potential antagonists include small organic molecules, peptides, polypeptides and antibodies that bind to neurons at the same site as CyPA and thus prevent the binding of CyPA, and the biological effects it confers. Potential antagonists also may be small organic molecules, a peptide, a polypeptide such as a closely related protein or antibody that binds to an alternative site on the neuron and prevents the action of CyPA by excluding polypeptide binding.

Thus, the present invention also provides a method for screening a compound for neuroactivity comprising contacting a candidate with CD147 and assessing binding and or receptor signalling.

The compounds which may be screened in accordance with the invention include, but are not limited to peptides, antibodies and fragments thereof, and other organic compounds (e.g., peptidomimetics). Useful compounds found using the screen may either mimic the activity triggered by CyPA (i.e., agonists) and thus be useful as neuroprotectants or inhibit the activity triggered by CyPA (i.e., antagonists).

Computer modelling and searching technologies permit identification of candidates, or the improvement of already identified candidates that can bind and/or evoke CD147 receptor signalling. Having identified such candidates, the active sites or regions are identified. Such active sites might typically be ligand binding sites, such as the interaction domains of CyPA with CD147 itself. The active site can be identified using methods known in the art including, for example, from study of complexes of CyPA with CD147. In this regard, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances.

Having determined the structure of the active site, either experimentally, by modelling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential neuroactive compounds.

Alternatively, these methods can be used to identify improved neuroactive compounds from an already known neuroactive compound. The known compound can be modified and the structural effects of modification can be determined using the experimental and computer modelling methods described above applied to the new composition. The altered structure is then compared to the active site structure of the compound to determine if an improved fit or interaction results. In this manner systematic variations in composition, such as by varying side groups, can be quickly evaluated to obtain modified neuroactive compounds of improved specificity or activity.

Further experimental and computer modelling methods useful to identify neuroactive compounds based upon identification of the active sites of CyPA and CD147 will be apparent to those of skill in the art.

In vitro systems may be designed to identify compounds capable of interacting with (e.g., binding to) CD147 (including, but not limited to, the extra cellular domain of CD147). These compounds may be useful, for example, in modulating the activity of wild type and/or mutant CD147; elaborating the biological function of CD147; screening for compounds that disrupt normal CD147 interactions; or may in themselves disrupt such interactions. Alternatively, animal stroke models may be used to screen for functional variants.

The principle of the assays used to identify compounds that bind to CD147 involves preparing a reaction mixture of the CD147 and the candidate compound under conditions and for a time sufficient to allow the two components to interact and bind, thus forming a complex which can be removed and/or detected in the reaction mixture. The CD147 species used can vary depending upon the goal of the screening assay. For example, where agonists of CyPA are sought, the full length CD147, or a soluble truncated CD147, e.g., in which the transmembrane or cellular domain is deleted from the molecule, a peptide corresponding to the extracellular domain or a fusion protein comprising the CD147 extracellular domain fused to a protein or polypeptide that affords advantages in the assay system (e.g., labelling, isolation of the resulting complex, etc.) can be utilized.

The screening assays can be conducted in a variety of ways. For example, one method to conduct such an assay involves anchoring CD147 or a fusion protein thereof or the candidate onto a solid phase and detecting CD147/candidate complexes anchored on the solid phase at the end of the reaction. In one embodiment of such a method, the CD147 may be anchored onto a solid surface, and the test compound, which is not anchored, may be labelled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the CD147 or candidate and drying. Alternatively, an immobilized antibody, such as a monoclonal antibody, specific for the protein to be immobilized may be used to anchor the protein to the solid surface.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labelled, the detection of label immobilized on the surface indicates that complexes were formed. Where the previously nonimmobilized component is not pre-labelled, an indirect label can be used to detect complexes anchored on the surface; e.g., using a labelled antibody specific for the previously nonimmobilized component (the antibody, in turn, may be directly labelled or indirectly labelled with a labelled anti-Ig antibody).

Alternatively, a reaction can be conducted in a liquid phase, the reaction products separated from unreacted components, and complexes detected; e.g., using an immobilized antibody specific for CD147 or the candidate to anchor any complexes formed in solution, and a labelled antibody specific for the other component of the possible complex to detect anchored complexes.

Cell-based assays can also be used to identify compounds that interact with CD147. To this end, cell lines that express CD147 can be used. Interaction of the candidate with, for example, the extracellular domain of CD147 expressed by the host cell can be determined by comparison or competition with native CyPA.

Given the role of CD147, CyPA and functional variants thereof in conferring neuroprotection, it may also be possible to screen patients who may be predisposed to poor outcomes from conditions characterized by cerebral ischemia, such as stroke; and other conditions such as Alzheimer's disease, Parkinson's Disease, Motor Neurone Disease, any neurodegeneration and neuronal loss due to trauma and spinal cord damage, Huntington's disease, traumatic brain injury, multiple sclerosis, epilepsy, ischemic optic neuropathy and retinal degeneration disorders.

Thus, the present invention also provides a screening method comprising the steps of: (i) detecting the presence and/or measuring the level at least one of CD147, CyPA or a functional variant thereof in a patient; and (ii) comparing the result from (i) with a reference measure indicative of normality.

### Methods of Controlling Neural Degeneration

The present invention provides a method for controlling neural degeneration comprising the step of contacting a neuron with an effective amount of CyPA or a functional equivalent thereof.

The control of neural degeneration includes reduction and removal of neural degeneration. Thus, the present invention covers the use of CyPA or a functional equivalent thereof as a partial or complete neuroprotectant.

There are many disorders associated with neural degeneration and the activity of CyPA and functional equivalents thereof renders them useful as treatment options. Thus, the present invention also provides a method for treating a disease or disorder associated with neural degeneration comprising the step of administering to a subject an effective amount of CyPA or a functional equivalent thereof.

The disease or disorder may be selected from the group consisting of: conditions characterized by cerebral ischemia, such as stroke; and other conditions characterized by cerebral ischemia, such as stroke; and other conditions such as Alzheimer's disease, Parkinson's Disease, Motor Neurone Disease, any neurodegeneration and neuronal loss due to trauma and spinal cord damage, Huntington's disease, traumatic brain injury, multiple sclerosis, epilepsy, ischemic optic neuropathy and retinal degeneration disorders.

The neuroprotective polypeptide may be administered as a therapeutic or a prophylactic depending on the particular circumstances and as deemed appropriate by a medical practitioner.

Thus, the present invention also provides for the prophylactic use of CyPA or a functional variant thereof to reduce or prevent neuronal degeneration such as that caused by a disease or disorder selected from the group consisting of: conditions characterized by cerebral ischemia, such as stroke; and other conditions such as Alzheimer's disease, Parkinson's Disease, Motor Neurone Disease, any neurodegeneration and neuronal loss due to trauma and spinal cord damage, Huntington's disease, traumatic brain injury, multiple sclerosis, epilepsy, ischemic optic neuropathy and retinal degeneration disorders.

The effect of the administered therapeutic composition can be monitored by standard diagnostic procedures. For example, in the treatment of the neurodegeneration that follows a stroke, the administration of a composition that includes neuroprotective peptides can reduce the degeneration of CA1 hippocampal neurons. The reduction of degeneration of CA1 hippocampal neurons following treatment can be assessed using MRI and CT scans. Where other indicia of neurodegeneration are available, such indicia may also be used in diagnosing neurodegeneration following treatment with the polypeptide compositions.

Thus, the present invention also provides a method for reducing the degeneration of CA1 hippocampal neurons comprising the step of contacting the neuron with an effective amount of CyPA or a functional equivalent thereof.

### Pharmaceutical Compositions

This invention also provides pharmaceutical or veterinary compositions comprising CyPA or a functional variant thereof and a pharmaceutically acceptable carrier.

Pharmaceutical compositions of proteaceous drugs of this invention are particularly useful for parenteral administration, i.e., subcutaneously, intramuscularly or intravenously. The compositions for parenteral administration will commonly comprise a solution of the compounds of the invention or a cocktail thereof dissolved in an acceptable carrier, preferably an aqueous carrier. A variety of aqueous carriers may be employed, e.g., water, buffered water, 0.4% saline, 0.3% glycine, and the like. These solutions are sterile and generally free of particulate matter. These solutions may be sterilized by conventional, well known sterilization techniques. The compositions may further contain pharmaceutically acceptable auxiliary substances as required to approximate physiological conditions such as pH adjusting and buffering agents, etc.

The concentration of the compounds of the invention in such pharmaceutical formulation can very widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 ml sterile buffered water, and 50 mg of a compound of the invention. Similarly, a pharmaceutical composition of the invention for intravenous infusion could be made up to contain 250 ml of sterile Ringer's solution, and 150 mg of a compound of the invention. Actual methods for preparing parenterally administrable compositions are well known or will be apparent to those skilled in the art and are described in more detail in, for example, Remington's Pharmaceutical Science, 15th ed., Mack Publishing Company, Easton, Pa.

The compounds described herein can be lyophilized for storage and reconstituted in a suitable carrier prior to use. This technique has been shown to be effective with conventional proteins and art-known lyophilization and reconstitution techniques can be employed.

In situations where the functional variant is non-proteinaceous, it may be administered alone or in combination with pharmaceutically acceptable carriers. The proportion of the constituents in any formulation is determined by the solubility and chemical nature of the compound, chosen route of administration and standard pharmaceutical practice. For example, they may be administered orally in the form of tablets or capsules containing such excipients as starch, milk sugar, certain types of clay and so forth. They may be administered sublingually in the form of troches or lozenges in which the active ingredient is mixed with sugar and corn syrups, flavouring agents and dyes; and then dehydrated sufficiently to make it suitable for pressing into a solid form. They may be administered orally in the form of solutions that may be injected parenterally, that is, intramuscularly, intravenously or subcutaneously. For parenteral administration, they may be used in the form of a sterile solution containing other solutes, for example, enough saline or glucose to make the solution isotonic.

The physician or veterinarian will determine the dosage of the present therapeutic agents that will be most suitable and it will vary with the form of administration and the particular compound chosen, and furthermore, it will vary with the particular subject under treatment. The physician will generally wish to initiate treatment with small dosages substantially less than the optimum dose of the compound and increase the dosage by small increments until the optimum effect under the circumstances is reached. It will generally be found that when the composition is administered orally, larger quantities of the active agent will be required to produce the same effect as a smaller quantity given parenterally. The compounds are useful in the same manner as other serotonergic agents and the dosage level is of the same order of magnitude as is generally employed with these other therapeutic agents. The therapeutic dosage will generally be from 1 to 10 milligrams per day and higher although it may be administered in several different dosage units. Tablets containing from 0.5 to 10 mg of active agent are particularly useful.

As indicated above and depending on the subject's condition, the compositions of the invention can be administered for prophylactic and/or therapeutic treatments. In therapeutic application, compositions are administered to a subject suffering from an event associated with neuronal degeneration and/or involving ischemia in an amount sufficient to overcome the neuronal implications of the event. In prophylactic applications, compositions containing the CyPA or a functional variant thereof are administered to a subject predisposed to a condition associated with neuronal degeneration such as an ischemic event to reduce the damage suffered by the subject during the event.

Single or multiple administrations of the compositions can be carried out with dose levels and pattern being selected by the treating physician or veterinarian. In any event, the composition of the invention should provide a quantity of the compounds of the invention sufficient to effectively treat the subject.

The term “pharmaceutically acceptable” means a non-toxic material that does not interfere with the effectiveness of the biological activity of the active ingredients. The characteristics of the carrier will depend on the route of administration. Pharmaceutically acceptable carriers include diluents, fillers, salts, buffers, stabilizers, solubilizers, and other materials that are well known in the art.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating conditions characterized by recurrent cerebral ischemia, thereby affecting localized, high-doses of the compounds of the invention.

The present invention also provides for the use of CyPA or a functional variant thereof to prepare a medicament for treating or preventing neuronal degeneration or a disease or disorder characterized by cerebral ischemia, such as stroke; and other conditions such as Alzheimer's disease, Parkinson's Disease, Motor Neurone Disease, any neurodegeneration and neuronal loss due to trauma and spinal cord damage, Huntington's disease, traumatic brain injury, multiple sclerosis, epilepsy, ischemic optic neuropathy and retinal degeneration disorders.

### Antibodies

This invention also provides antibodies, monoclonal or polyclonal directed to epitopes of the peptides disclosed herein. Particularly important regions of the peptides for immunological purposes are those regions associated with ligand binding domains of the protein. Antibodies directed to these regions are particularly useful in diagnostic and therapeutic applications because of their effect upon protein-ligand interaction. Methods for the production of polyclonal and monoclonal antibodies are well known amongst those skilled in the art.

This invention also provides pharmaceutical compositions comprising an effective amount of antibody or fragment thereof directed against a polypeptide described herein to block its binding.

The polypeptides of the present invention or their fragments comprising at least one epitope can be used to produce antibodies, both polyclonal and monoclonal. If polyclonal antibodies are desired, a selected mammal, (e.g., mouse, rabbit, goat, horse, etc.) is immunized with a polypeptide of the present invention, or its fragment, or a mutated binding protein. Serum from the immunized animal is collected and treated according to known procedures. When serum containing polyclonal antibodies is used, the polyclonal antibodies can be purified by immunoaffinity chromatography or other known procedures.

Monoclonal antibodies to the polypeptides of the present invention, and to the fragments thereof, can also be readily produced by one skilled in the art. The general methodology for making monoclonal antibodies by using hybridoma technology is well known. Immortal antibody-producing cell lines can be created by cell fusion, and also by other techniques such as direct transformation of B lymphocytes with oncogenic DNA, or transfection with Epstein-Barr virus.

Panels of monoclonal antibodies produced against the protein of interest, or fragment thereof, can be screened for various properties; i.e., for isotype, epitope, affinity, etc. Alternatively, genes encoding the monoclonals of interest may be isolated from the hybridomas by PCR techniques known in the art and cloned and expressed in the appropriate vectors. Monoclonal antibodies are useful in purification, using immunoaffinity techniques, of the individual proteins against which they are directed. The antibodies of this invention, whether polyclonal or monoclonal have additional utility in that they may be employed as reagents in immunoassays, RIA, ELISA, and the like.

### Polynucleotides

The present invention also provides an isolated polynucleotide encoding CypA or a functional variant thereof.

Polynucleotides of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” polynucleotide(s) is intended a polynucleotide, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution.

Isolated RNA molecules include in vivo or in vitro RNA transcripts of the DNA molecules of the present invention. Isolated polynucleotides according to the present invention further include such molecules produced synthetically.

Polynucleotides of the present invention include those that comprise a nucleotide sequence different to those explicitly described herein but which, due to the degeneracy of the genetic code, still encode the same polypeptide. Of course, the genetic code is well known in the art. Thus, it would be routine for one skilled in the art to generate such degenerate variants of the polynucleotides of the present invention.

The present invention also provides fragments of the polynucleotides of the present invention. Preferred fragments comprise at least 10, 20, 30, 40, 50, 60 or 70 contiguous nucleotides. Other preferred fragments encode polypeptides with at least one important property of the full length polypeptide or epitope bearing portions of the larger polypeptide. Methods for determining fragments would be readily apparent to one skilled in the art and are exemplified in more detail below.

The polynucleotides of the present invention may be used in accordance with the present invention for a variety of applications, particularly those that make use of the chemical and biological properties of CyPA.

The present invention also provides isolated polynucleotides that selectively hybridize with at least a portion of a polynucleotide of the present invention. As used herein to describe nucleic acids, the term “selectively hybridize” excludes the occasional randomly hybridizing nucleic acids under at least moderate stringency conditions. Thus, selectively hybridizing polynucleotides preferably hybridize under at least moderate stringency conditions and more preferably under high stringency conditions. The hybridising polynucleotides may be used, for example, as probes or primers for detecting the presence of polynucleotides encoding CyPA such as cDNA or mRNA.

A nucleic acid molecule is “hybridizable” to another nucleic acid molecule, such as a cDNA, genomic DNA, or RNA, when a single-stranded form of the nucleic acid molecule can anneal to the other nucleic acid molecule under the appropriate conditions of temperature and solution ionic strength. The conditions of temperature and ionic strength determine the “stringency” of the hybridization. For preliminary screening for homologous nucleic acids, low stringency hybridization conditions, corresponding to a Tₘ of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived and are known to those skilled in the art. For hybridization with shorter nucleic acids, i.e., oligonucleotides, the position of mismatches becomes more important, and the length of the oligonucleotide determines its specificity. Preferably a minimum length for a hybridizable nucleic acid is at least about 10 nucleotides; more preferably at least about 15 nucleotides; most preferably the length is at least about 20, 30 or 40-70 nucleotides.

Of course, a polynucleotide which hybridizes only to a poly A sequence (such as a 3′ terminal poly(A) tail of a polynucleotide of the present invention), or to a complementary stretch of T (or U) resides, would not be included as a selectively hybridizable polynucleotide of the invention, since such a polynucleotide would hybridize to any nucleic acid molecule containing a poly (A) stretch or the complement thereof (e.g., practically any double-stranded cDNA clone).

Using the nucleic acid sequences taught herein and relying on cross-hybridization, one skilled in the art can identify polynucleotides in other species that encode polypeptides of the invention. If used as primers, the invention provides compositions including at least two nucleic acids that selectively hybridize with different regions of the target nucleic acid so as to amplify a desired region. Depending on the length of the probe or primer, the target region can range between 70% complementary bases and full complementarity.

The selectively hybridisable polynucleotides described herein or more particularly portions thereof can be used to detect the nucleic acid of the present invention in samples by methods such as the polymerase chain reaction, ligase chain reaction, hybridization, and the like. Alternatively, these sequences can be utilized to produce an antigenic protein or protein portion, or an active protein or protein portion.

In addition, portions of the selectively hybridisable polynucleotides described herein can be selected to selectively hybridize with homologous polynucleotides in other organisms. These selectively hybridisable polynucleotides can be used, for example, to simultaneously detect related sequences for cloning of homologues of the polynucleotides of the present invention.

As indicated above, the polynucleotides of the present invention that encode a polypeptide of the present invention include, but are not limited to, those encoding the amino acid sequence of the polypeptide, by itself. Rather the polynucleotides of the present invention may comprise the coding sequence for the polypeptide and additional sequences, such as those encoding a leader or secretory sequence, such as a pre-, or pro- or prepro-protein sequence; the coding sequence of the polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example ribosome binding and stability of mRNA; an additional coding sequence which codes for additional amino acids, such as those which provide additional functionalities. Polynucleotides according to the present invention also include those encoding a polypeptide, such as the entire protein, lacking the N terminal methionine.

Thus, polynucleotides of the present invention include those with a sequence encoding a polypeptide of the invention fused to a marker sequence, such as a sequence encoding a peptide that facilitates purification of the fused polypeptide. In certain preferred embodiments of this aspect of the invention, the marker amino acid sequence is a hexa histidine peptide, such as the tag provided in a pQE vector (Qiagen, Inc.), among others, many of which are commercially available. The “HA” tag is another peptide useful for purification which corresponds to an epitope derived from the influenza hemagglutinin protein.

The present invention further relates to variants of the nucleic acid molecules of the present invention, which encode portions, analogs or derivatives of the polypeptides of the present invention. Variants may occur naturally, such as a natural allelic variant. By an “allelic variant” is intended one of several alternate forms of a gene occupying a given locus on a chromosome of an organism. Non-naturally occurring variants may be produced using mutagenesis techniques known to those in the art.

Such variants include those produced by nucleotide substitutions, deletions or additions that may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the encoded polypeptide. Also especially preferred in this regard are conservative substitutions.

The present invention also includes isolated polynucleotides comprising a nucleotide sequence at least 60, 70, 80 or 90% identical, and more preferably at least 95%, 96%, 97%, 98% or 99% identical to a nucleotide sequence encoding the polypeptide having the complete amino acid sequence in SEQ ID NO: 2 or 4.

For the purposes of the present invention a nucleotide sequence that is 95% identical to a reference sequence is identical to the reference sequence except that it may include up to five point mutations per each 100 nucleotides of the reference nucleotide sequence. In other words, to obtain a polynucleotide having a nucleotide sequence at least 95% identical to reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

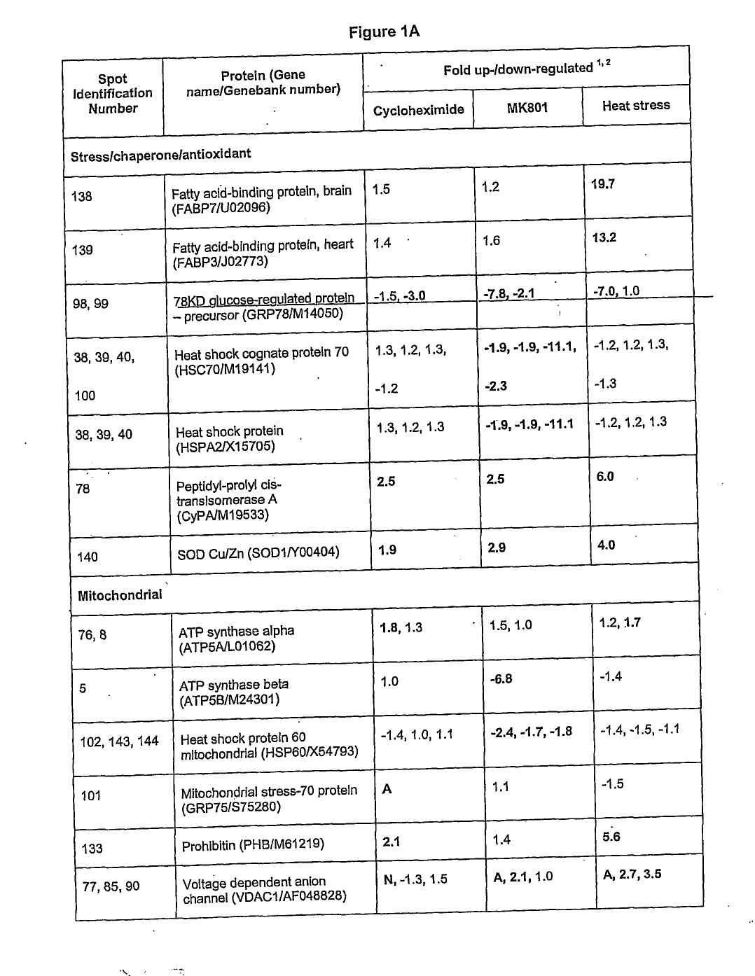

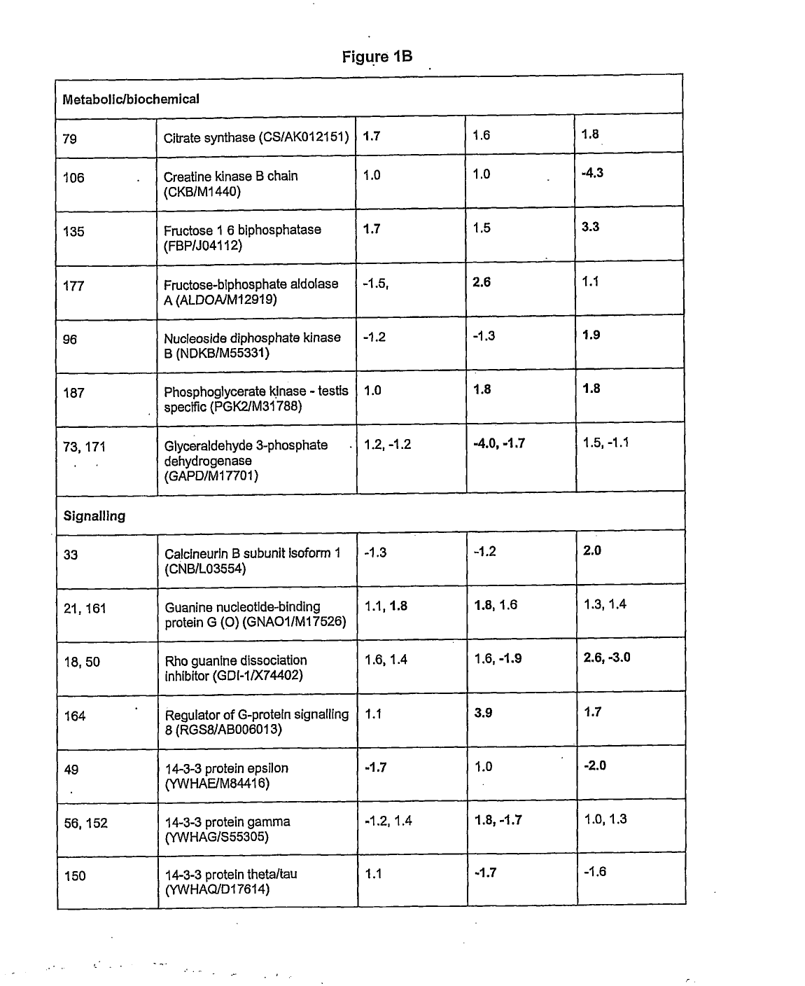

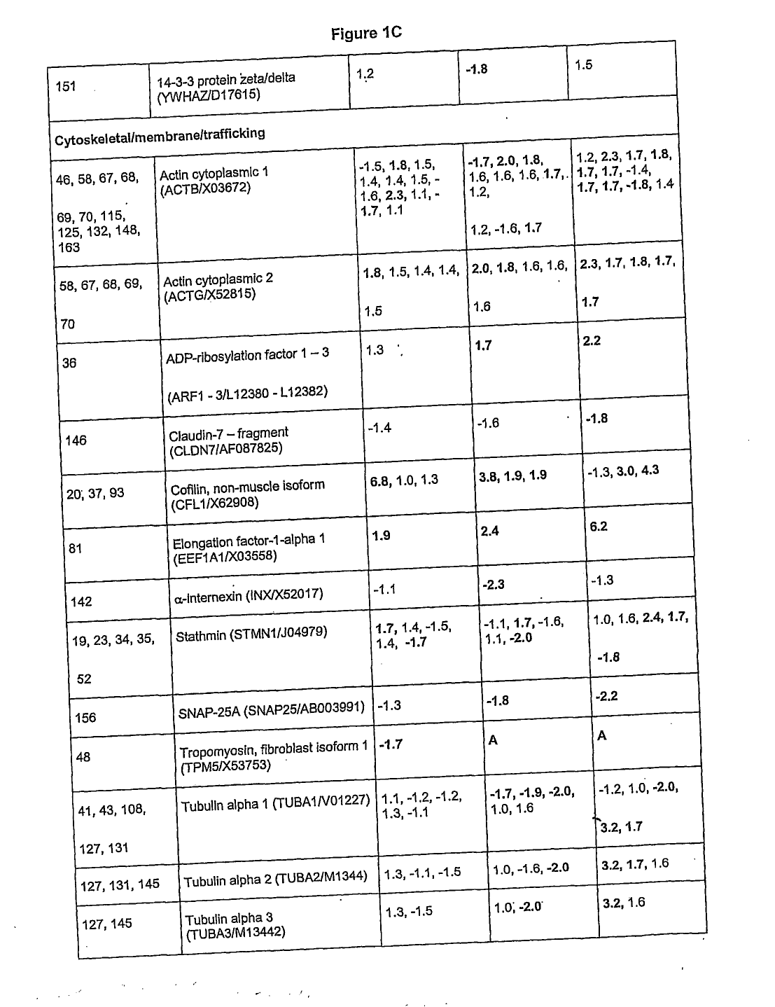

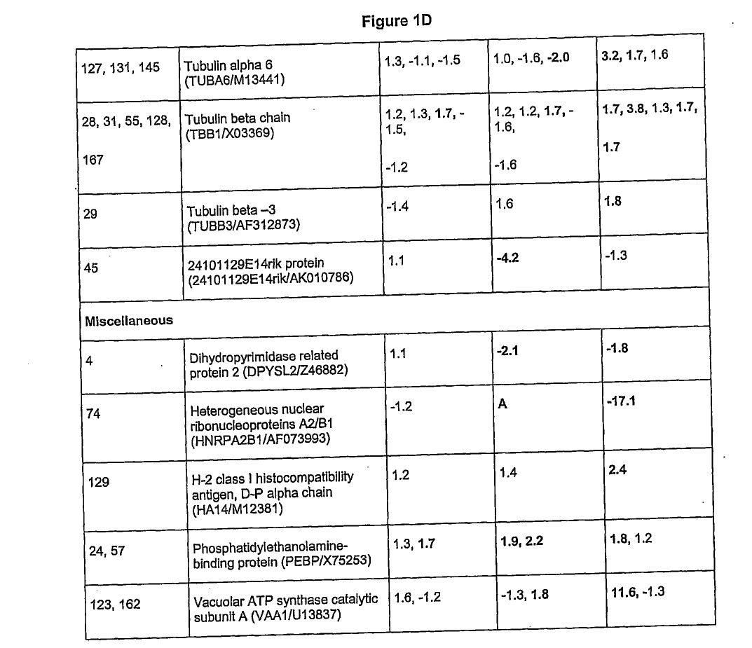

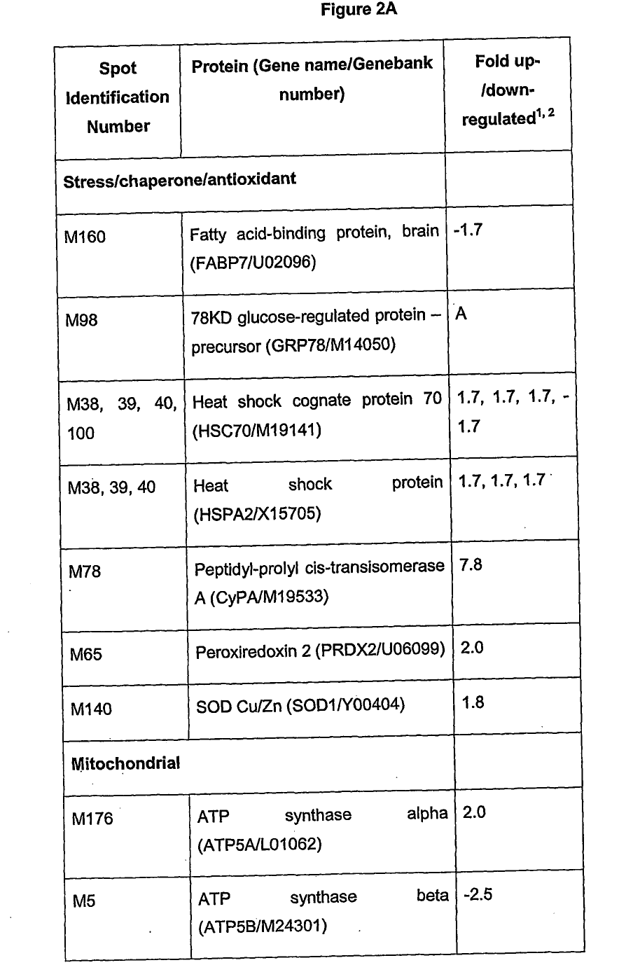

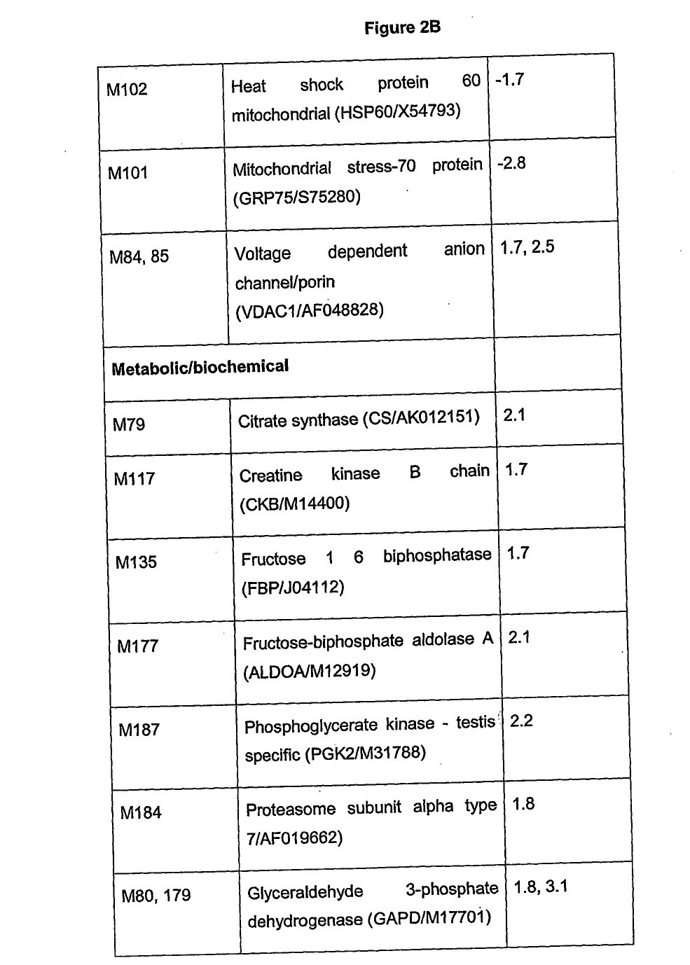

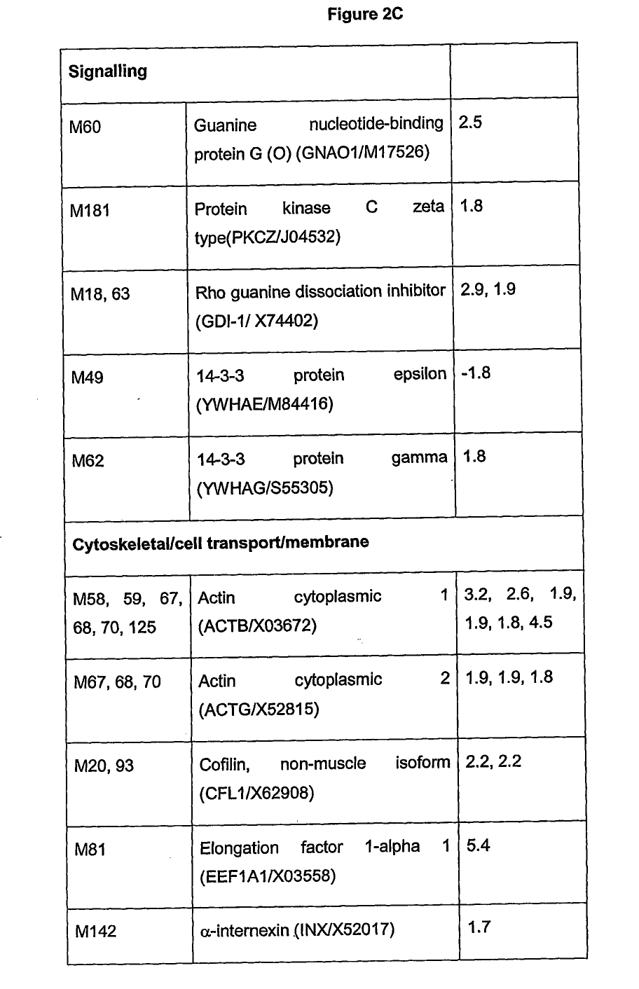

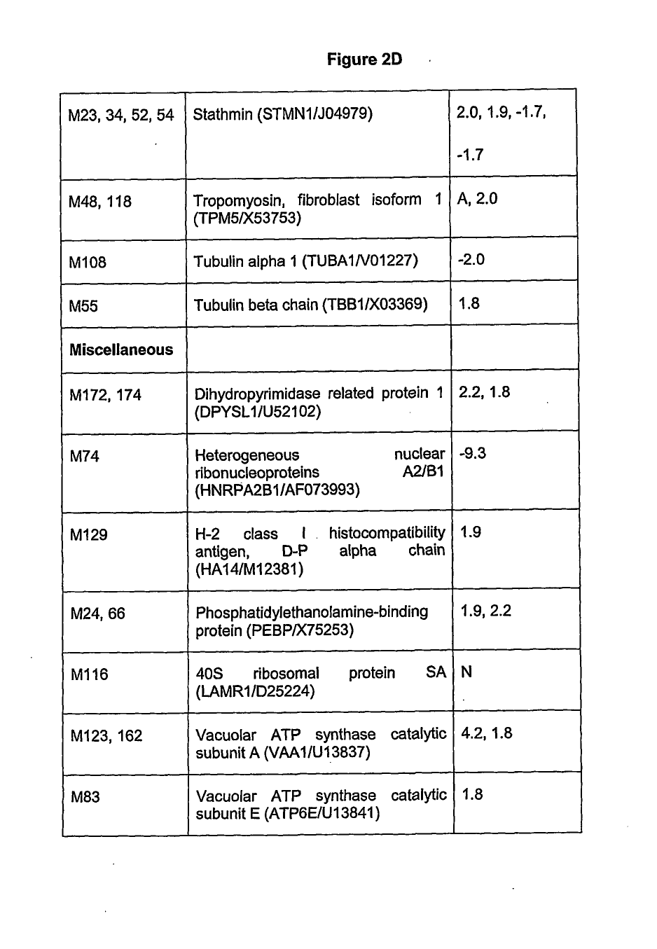

As a practical matter, whether any particular nucleic acid molecule is at least 60, 70, 80, 90%, 95%, 96%, 97%, 98% or 99% 90%, 95%, 96%, 97%, 98% or 99% identical to, for instance, the nucleotide sequence encoding a polypeptide in FIG. 1 or 2 or can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

Of course, due to the degeneracy of the genetic code, one of ordinary skill in the art will immediately recognize that a large number of the nucleic acid molecules having a sequence at least 60, 70, 80, 90, 95, 96, 97, 98 or 99 identical to the nucleic acid sequence of the polypeptides in FIG. 1 or 2 will encode a polypeptide within the scope of the present invention. In fact, since degenerate variants of these nucleotide sequences all encode the same polypeptide, this will be clear to the skilled artisan even without performing the above described comparison.

It will be further recognized in the art that, for such nucleic acid molecules that are not degenerate variants, a reasonable number will also encode a polypeptide having ML binding activity. This is because the skilled artisan is fully aware of amino acid substitutions that are either less likely or not likely to significantly affect protein function (e.g., replacing one aliphatic amino acid with a second aliphatic amino acid).

### Gene/Cell Therapy

The CyPA or functional variant thereof can be delivered by implanting certain cells that have been genetically engineered, using methods such as those described herein, to express and secrete the polypeptide of interest. Such cells may be animal or human cells, and may be autologous, heterologous, or xenogenic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally silent transcriptionally silent gene encoding a polypeptide described herein, or an under expressed gene, and thereby produce a cell which expresses therapeutically efficacious amounts of the polypeptides.

Homologous recombination is a technique originally developed for targeting genes to induce or correct mutations in transcriptionally active genes. The basic technique was developed as a method for introducing specific mutations into specific regions of the mammalian genome or to correct specific mutations within defective genes. Through homologous recombination, a given DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process.

It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome. Attached to these pieces of targeting DNA are regions of DNA that may interact with or control the expression of a polypeptide herein, e.g., flanking sequences. For example, a promoter/enhancer element, a suppresser or an exogenous transcription modulatory element is inserted in the genome of the intended host cell in proximity and orientation sufficient to influence the transcription of DNA encoding the desired polypeptide. The control element controls a portion of the DNA present in the host cell genome. Thus, the expression of the desired polypeptide of the present invention may be achieved not by transfection of DNA that encodes the polypeptide itself, but rather by the use of targeting DNA (containing regions of homology with the endogenous gene of interest), coupled with DNA regulatory segments that provide the endogenous gene sequence with recognizable signals for transcription of the gene encoding the polypeptide.

In an exemplary method, the expression of a gene encoding CyPA or a functional variant thereof in a cell (i.e., a desired endogenous cellular gene) is altered via homologous recombination into the cellular genome at a preselected site, by the introduction of DNA that includes at least a regulatory sequence, an exon and a splice donor site. These components are introduced into the chromosomal (genomic) DNA in such a manner that this, in effect, results in the production of a new transcription unit (in which the regulatory sequence, the exon and the splice donor site present in the DNA construct are operatively linked to the endogenous gene). As a result of the introduction of these components into the chromosomal DNA, the expression of the desired endogenous gene is altered.

Altered gene expression, as described herein, encompasses activating (or causing to be expressed) a gene which is normally silent (unexpressed) in the cell, as well as increasing the expression of a gene which is not expressed at physiologically significant levels in the cell. The embodiments further encompass changing the pattern of regulation or induction such that it is different from the pattern of regulation or induction that occurs in the cell, and reducing (including eliminating) the expression of a gene which is expressed in the cell.

One method by which homologous recombination can be used to increase, or cause production of a polypeptide described herein from a cell's endogenous gene involves first using homologous recombination to place a recombination sequence from a site-specific recombination system (e.g., Cre/IoxP, FLP/FRT) (see, Sauer, Current Opinion In Biotechnology, 5:521-527, 1994; and Sauer, Methods In Enzymology, 225:890-900, 1993) upstream (that is, 5′ to) of the cell's endogenous genomic polypeptide coding region. A plasmid containing a recombination site homologous to the site that was placed just upstream of the genomic polypeptide coding region is introduced into the modified cell line along with the appropriate recombinase enzyme. This recombinase enzyme causes the plasmid to integrate, via the plasmid's recombination site, into the recombination site located just upstream of the genomic polypeptide coding region in the cell line (Baubonis and Sauer, Nucleic Acids Res., 21:2025-2029, 1993; and O'Gorman et al., Science, 251: 1351-1355, 1991). Any flanking sequences known to increase transcription (e.g., enhancer/promoter, intron or translational enhancer), if properly positioned in this plasmid, would integrate in such a manner as to create a new or modified transcriptional unit resulting in de novo or increased polypeptide production from the cell's endogenous gene.

A further method to use the cell line in which the site-specific recombination sequence has been placed just upstream of the cell's endogenous genomic polypeptide coding region is to use homologous recombination to introduce a second recombination site elsewhere in the cell line's genome. The appropriate recombinase enzyme is then introduced into the two-recombination-site cell line, causing a recombination event (deletion, inversion or translocation) (Sauer, Current Opinion In Biotechnology, supra, 1994 and Sauer, Methods In Enzymology, supra, 1993) that would create a new or modified transcriptional unit resulting in de novo or increased polypeptide production from the cell's endogenous gene.

Another approach for increasing, or causing, the expression of the polypeptide from a cell's endogenous gene involves increasing, or causing, the expression of a gene or genes (e.g., transcription factors) and/or decreasing the expression of a gene or genes (e.g., transcriptional repressors) in a manner which results in de novo or increased polypeptide production from the cell's endogenous gene. This method includes the introduction of a non-naturally occurring polypeptide (e.g., a polypeptide comprising a site-specific DNA binding domain fused to a transcriptional factor domain) into the cell such that de novo or increased polypeptide production from the cell's endogenous gene results.

The present invention further relates to DNA constructs useful in the method of altering expression of a target gene. In certain embodiments, the exemplary DNA constructs comprise: (a) one or more targeting sequences; (b) a regulatory sequence; (c) an exon; and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence of the polypeptides presented herein, a piece of DNA that is complementary to a selected region of the gene can be synthesized or otherwise obtained, such as by appropriate restriction of the native DNA at specific recognition sites bounding the region of interest. This piece serves as a targeting sequence(s) upon insertion into the cell and will hybridize to its homologous region within the genome. If this hybridization occurs during DNA replication, this piece of DNA, and any additional sequence attached thereto, will act as an Okazaki fragment and will be incorporated into the newly synthesized daughter strand of DNA. The present invention, therefore, includes nucleotides encoding a polypeptide, which nucleotides may be used as targeting sequences.

Polypeptide cell therapy, e.g., the implantation of cells producing polypeptides described herein, is also contemplated. This embodiment involves implanting cells capable of synthesizing and secreting a biologically active form of the polypeptide. Such polypeptide-producing cells can be cells that are natural producers of the polypeptides or may be recombinant cells whose ability to produce the polypeptides has been augmented by transformation with a gene encoding the desired polypeptide or with a gene augmenting the expression of the polypeptide. Such a modification may be accomplished by means of a vector suitable for delivering the gene as well as promoting its expression and secretion. In order to minimize a potential immunological reaction in patients being administered a polypeptide, as may occur with the administration of a polypeptide of a foreign species, it is preferred that the natural cells producing polypeptide be of human origin and produce human polypeptide. Likewise, it is preferred that the recombinant cells producing polypeptide be transformed with an expression vector containing a gene encoding a human polypeptide.

Implanted cells may be encapsulated to avoid the infiltration of surrounding tissue. Human or non-human animal cells may be implanted in patients in biocompatible, semipermeable polymeric enclosures or in membranes that allow the release of polypeptide, but prevent the destruction of the cells by the patient's immune system or by other detrimental factors from the surrounding tissue. Alternatively, the patient's own cells, transformed to produce polypeptides ex vivo, may be implanted directly into the patient without such encapsulation.

Techniques for the encapsulation of living cells are known in the art, and the preparation of the encapsulated cells and their implantation in patients may be routinely accomplished. For example, Baetge et al. (WO 95/05452 and PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down-regulation in vivo upon implantation into a mammalian host. The devices provide for the delivery of the molecules from living cells to specific sites within a recipient. A system for encapsulating living cells is described in PCT Application PCT/US91/00157 of Aebischer et al. See also, PCT Application PCT/US91/00155 of Aebischer et al.; Winn et al., Exper. Neurol., 113:322-329 (1991), Aebischer et al., Exper. Neurol, 111:269-275 (1991); and Tresco et al., ASAIO, 38:17-23 (1992).

In vivo and in vitro gene therapy delivery of polypeptides is also part of the present invention. One example of a gene therapy technique is to use the gene (either genomic DNA, cDNA, and/or synthetic DNA) encoding a polypeptide described herein that may be operably linked to a constitutive or inducible promoter to form a “gene therapy DNA construct”. The promoter may be homologous or heterologous to the endogenous gene, provided that it is active in the cell or tissue type into which the construct will be inserted. Other components of the gene therapy DNA construct may optionally include, DNA molecules designed for site-specific integration (e.g., endogenous sequences useful for homologous recombination); tissue-specific promoter, enhancer(s) or silencer(s); DNA molecules capable of providing a selective advantage over the parent cell; DNA molecules useful as labels to identify transformed cells; negative selection systems, cell specific systems; cell-specific binding agents (as, for example, for cell targeting); cell-specific internalization factors; and transcription factors to enhance expression by a vector, as well as factors to enable vector manufacture.

A gene therapy DNA construct can then be introduced into cells (either ex vivo or in vivo) using viral or non-viral vectors. Certain vectors, such as retroviral vectors, will deliver the DNA construct to the chromosomal DNA of the cells, and the gene can integrate into the chromosomal DNA. Other vectors will function as episomes, and the gene therapy DNA construct will remain in the cytoplasm.

In yet other embodiments, regulatory elements can be included for the controlled expression of the gene in the target cell. Such elements are turned on in response to an appropriate effector. In this way, a therapeutic polypeptide can be expressed when desired. One conventional control means involves the use of small molecule dimerizers or rapalogs (as described in WO 9641865 (PCT/US96/099486); WO 9731898 (PCT/US97/03137) and WO9731899 (PCT/US95/03157) used to dimerize chimeric proteins which contain a small molecule-binding domain and a domain capable of initiating biological process, such as a DNA-binding protein or a transcriptional activation protein. The dimerization of the proteins can be used to initiate transcription of the transgene.

An alternative regulation technology uses a method of storing proteins expressed from the gene of interest inside the cell as an aggregate or cluster. The gene of interest is expressed as a fusion protein that includes a conditional aggregation domain that results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins may be secreted from the cell.

Another control means uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide that activates transcription.

In vivo gene therapy may be accomplished by introducing the gene encoding a polypeptide into cells via local injection of a nucleic acid molecule or by other appropriate viral or non-non-viral delivery vectors. For example, a nucleic acid molecule encoding a polypeptide of the present invention may be contained in an adeno-associated virus (AAV) vector for delivery to the targeted cells (e.g., Johnson, International Publication No. WO95/34670; and International Application No. PCT/US95/07178). The recombinant AAV genome typically contains MV inverted terminal repeats flanking a DNA sequence encoding a polypeptide operably linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells that have been treated in vitro to insert a DNA segment encoding a therapeutic protein. Additional methods and materials for the practice of gene therapy techniques are described in U.S. Pat. No. 5,631,236 involving adenoviral vectors; U.S. Pat. No. 5,672,510 involving retroviral vectors; and U.S. Pat. No. 5,635,399 involving retroviral vectors expressing cytokines.

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include the use of inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. No. 4,970,154 involving electroporation techniques; WO96/40958 involving nuclear ligands; U.S. Pat. No. 5,679,559 describing a lipoprotein-containing system for gene delivery; U.S. Pat. No. 5,676,954 involving liposome carriers; U.S. Pat. No. 5,593,875 concerning methods for calcium phosphate transfection; and U.S. Pat. No. 4,945,050 wherein biologically active particles are propelled at cells at a speed whereby the particles penetrate the surface of the cells and become incorporated into the interior of the cells.

It is also contemplated that gene therapy or cell therapy according to the present invention can further include the delivery of one or more additional polypeptide(s) in the same or a different cell(s). Such cells may be separately introduced into the patient, or the cells may be contained in a single implantable device, such as the encapsulating membrane described above, or the cells may be separately modified by means of viral vectors.

A means to increase endogenous polypeptide expression in a cell via gene therapy is to insert one or more enhancer element into the polypeptide promoter, where the enhancer element(s) can serve to increase transcriptional activity of the gene. The enhancer element(s) used will be selected based on the tissue in which one desires to activate the gene(s); enhancer elements known to confer promoter activation in that tissue will be selected. Here, the functional portion of the transcriptional element to be added may be inserted into a fragment of DNA containing the polypeptide promoter (and optionally, inserted into a vector and/or 5′ and/or 3′ flanking sequence(s), etc.) using standard cloning techniques. This construct, known as a “homologous recombination construct”, can then be introduced into the desired cells either ex vivo or in vivo.

Gene therapy also can be used to decrease polypeptide expression by modifying the nucleotide sequence of the endogenous promoter(s). Such modification is typically accomplished via homologous recombination methods. For example, a DNA molecule containing all or a portion of the promoter of the gene selected for inactivation can be engineered to remove and/or replace pieces of the promoter that regulate transcription. For example the TATA box and/or the binding site of a transcriptional activator of the promoter may be deleted using standard molecular biology techniques; such deletion can inhibit promoter activity thereby repressing the transcription of the corresponding gene. The deletion of the TATA box or the transcription activator binding site in the promoter may be accomplished by generating a DNA construct comprising all or the relevant portion of the polypeptide promoter(s) (from the same or a related species as the polypeptide gene to be regulated) in which one or more of the TATA box and/or transcriptional activator binding site nucleotides are mutated via substitution, deletion and/or insertion of one or more nucleotides. As a result, the TATA box and/or activator binding site has decreased activity or is rendered completely inactive. The construct will typically contain at least about 500 bases of DNA that correspond to the native (endogenous) 5′ and 3′ DNA sequences adjacent to the promoter segment that has been modified. The construct may be introduced into the appropriate cells (either ex vivo or in vivo) either directly or via a viral vector as described herein. Typically, the integration of the construct into the genomic DNA of the cells will be via homologous recombination, where the 5′ and 3′ DNA sequences in the promoter construct can serve to help integrate the modified promoter region via hybridization to the endogenous chromosomal DNA.

### Vectors, Host Cells and Expression

The polypeptides used in this invention are preferably made by recombinant genetic engineering techniques. The isolated polynucleotides, particularly the DNAs, can be introduced into expression vectors by operatively linking the DNA to the necessary expression control regions (e.g. regulatory regions) required for gene expression. The vectors can be introduced into appropriate host cells such as prokaryotic (e.g., bacterial), or eukaryotic (e.g., yeast or mammalian) cells by methods well known in the art.

The coding sequences for the polypeptides of the invention, having been prepared or isolated, can be cloned into any suitable vector or replicon. Numerous cloning vectors are known to those of skill in the art, and the selection of an appropriate cloning vector is a matter of choice. Examples of recombinant DNA vectors for cloning and host cells that they can transform include the bacteriophage lambda (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), a baculovirus insect cell system, YCp19 (Saccharomyces). See, generally, “DNA Cloning”: Vols. I & II, Glover et al., eds. IRL Press Oxford (1985) (1987) and; T. Maniatis et al. “Molecular Cloning”, Cold Spring Harbor Laboratory (1982).

The polynucleotides described herein can be placed under the control of a promoter (such as phage lambda PL promoter, the E. coli lac and trp promoters and the SV 40 early and late promoters), ribosome binding site (for bacterial expression) and, optionally, an operator (collectively referred to herein as “control” elements), so that the polynucleotide sequence encoding the polypeptide is transcribed into RNA in the host cell transformed by a vector containing the expression construction. The coding sequence may or may not contain a signal peptide or leader sequence.

The expression constructs may further contain sites for transcription initiation and termination. The coding portion of the mature transcripts expressed by the constructs will preferably include a translation initiating at the beginning and a termination codon (UAA, UGA or UAG) appropriately positioned at the end of the polypeptide to be translated.

In addition to control sequences, it may be desirable to add regulatory sequences that allow for regulation of the expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the particular protein of interest may be desirable to achieve this end. For example, in some cases it may be necessary to modify the sequence so that it may be attached to the control sequences with the appropriate orientation; i.e., to maintain the reading frame. The control sequences and other regulatory sequences may be ligated to the coding sequence prior to insertion into a vector, such as the cloning vectors described above. Alternatively, the coding sequence can be cloned directly into an expression vector that already contains the control sequences and an appropriate restriction site.

In some cases, it may be desirable to add sequences that cause the secretion of the polypeptide from the host organism, with subsequent cleavage of the secretory signal. Alternatively, gene fusions may be created whereby the gene encoding the polypeptide of the invention is fused to a gene encoding a product with other desirable properties. For example, a fusion partner could provide known assayable activity (e.g., enzymatic) that could be used as an alternative means of selecting the polypeptide. The fusion partner could also be a structural element, such as a cell surface element such that the polypeptide could be displayed on the cell surface in the form of a fusion protein. Alternatively, it could be peptide or protein fragment that can be detected with specific antibodies and reagents, and may act as an aid to purification (e.g. His tail, Glutathione S-transferase fusion).

The expression vectors may also include at least one selectable marker. Such markers include dihydrofolate reductase or neomycin resistance for eukaryotic cell culture and tetracycline or ampicillin resistance genes for culturing in E. coli and other bacteria.

It may also be desirable to produce mutants or analogs of the protein of interest. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis and the formation of fusion proteins, are well known to those skilled in the art.

Other representative examples of appropriate hosts include, but are not limited to, bacterial cells, such as E coli, Streptomyces and Salmonella typhimurium cells; fungal cells, such as yeast cells; insect cells such as Drosophila S2.

Depending on the expression system and host selected, the polypeptides of the present invention may be produced by growing host cells transformed by an expression vector described above under conditions whereby the polypeptide of interest is expressed. The polypeptide is then isolated from the host cells and purified. If the expression system secretes the polypeptide into growth media, the polypeptide can be purified directly from the media. If the polypeptide is not secreted, it can be isolated from cell lysates or recovered from the cell membrane fraction. The selection of the appropriate growth conditions and recovery methods are known to those skilled in the art.

### General

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### EXAMPLES

### Example 1

### Differential Protein Expression in Preconditioned Neuronal Cells

### Materials and Methods

### (1) Cultivation of Cortical Neurons

Establishment of cortical cultures was as previously described and briefly outlined below (Meloni et al, 2002).

Cortical tissue from E18-E19 rats was dissociated in Hibernate E medium (Invitrogen, Carlsbad, Calif., USA) supplemented with 1.3 mM L-cysteine, 10 units/ml papain (ICN, Costa Mesa, Calif., USA) and 50 units/ml DNase (Sigma, St. Louis, Mo., USA) and washed in cold Dulbecco's Modified Eagle Medium (Invitrogen)/10% horse serum.

Neurons were resuspended in Neurobasal (NB; Invitrogen)/2% B27 supplement (Invitrogen), the cell concentration was adjusted to 1.8 million neurons/2 ml and 2 ml inoculated into each well of a 6 well plate pretreated as described below.

Neuronal cultures were maintained in a CO₂ incubator (5% CO₂, 95% air balance, 98% humidity) at 37° C. On day in vitro (DIV) 4 one third of the culture medium was removed and replaced with fresh NB/2% B27 containing the mitotic inhibitor cytosine arabinofuranoside (final concentration 1 μM; Sigma), and on DIV 8 one half of the culture medium was replaced with NB/2% B27.

Neuronal cultures were exposed to preconditioning treatments on DIV 11. At DIV 11 between 1-2% of cells in neuronal cultures stain positively for glial fibrillary acidic protein.

### (2) Preparation of Culture Wells

Wells were coated with 700 μl of poly-D-lysine (40 μg/ml; 70-150K; Sigma) overnight at room temperature. The poly-D-lysine was removed and 1.25 ml of NB containing 2% B27, 4% fetal bovine serum, 1% horse serum, 62.5 μM glutamate, 25 μM 2-mercaptoethanol, 30 μg/ml penicillin and 50 μg/ml streptomycin was added to each well and incubated in a CO₂ incubator for 1-3 h before the addition of the 2 ml dissociated neuronal suspension.

### (3) Preconditioning Treatments

Heat stress (HS) preconditioning consisted of incubating neuronal cultures in a CO₂ incubator at 42.5° C. for 1 h and then returning cultures to the 37° C. CO₂ incubator for 24 hours. For cycloheximide (CHX; Sigma) preconditioning a concentrated stock of the agent was added to culture wells to achieve a final concentration of 0.3 μg/ml. Cycloheximide exposure was for 24 hours. We used a similar transient NMDA receptor inactivation method to that described by Tremblay et al. (2000 J. Neurosci. 20, 7183-7192). MK801 (1 μM; Tocris, Ballwin, Mo., USA) preconditioning was performed by, adding MK801 to wells and incubating at 37° C. for 30 min. MK801 was removed by two washes in balanced salt solution (BSS; mM: 116 NaCl, 5.4 KCl, 1.8 CaCl₂, 0.8 MgSO₄, 1 NaH₂PO₄; pH 7.3), one wash in conditioned media and reapplying conditioned medium to the wells before CO₂ incubation for 12 hours. Controls consisted of DIV 12 untreated cortical neuronal cultures.

### (4) Protein Isolation

Total protein was isolated from control and preconditioned neuronal cultures at the times outlined above by removing all the media from wells and washing once with phosphate-buffered saline, before the addition of lysis buffer (7M urea, 2M thiourea, 40 mM tris-HCl, 1% sulfobetaine 3-10, 2% CHAPS, 65 mM DTT, 1% Bio-Lyte carrier ampholytes pH 3-10; Bio-Rad, Hercules, Calif., USA). Sample was recovered from culture vessels and probe sonicated for 30 seconds (Branson Sonifier 450 constant duty cycle). Insoluble material was removed by centrifugation at 20,000 g for 10 minutes at room temperature. Samples were stored at −80° C. Protein content was determined by amino acid analysis using Waters AccQ Tag chemistry (Millipore Corporation, Milford, Mass., USA) as previously described (Cohen et al., 1983, in: Angeletti, R. H. (Ed.), Techniques in Protein Chemistry IV, Academic Press, San Diego).

### (5) 2-D Gel Electrophoresis

Two-dimensional electrophoresis was carried out on a Multiphor II flatbed electrophoresis system (Amersham Biosciences, Piscataway, N.J., USA) using 18 cm immobilised pH gradient (IPG) gel strips with pH ranges 4-7, 4.5-5.5 and 6-11 respectively (Amersham Biosciences). Sample corresponding to 80 μg protein was loaded onto pH 4-7 and pH 4.5-5.5 IPG strips via in-gel rehydration, while pH 6-11 strips were loaded at the anode using sample cups. Isoelectric focussing was carried out for a total of 95,000 V/hour at 20° C. Voltage was slowly increased from 300 V to 5000 V over 8 hours and maintained at 5000V until the final V/hour product was achieved. Each protein sample was run in triplicate.

Following isoelectric focussing strips were equilibrated for 30 minutes in 6M urea, 2% SDS, 20% glycerol, 0.375 M tris-HCl, pH 8.8, 5 mM tributylphosphine, 2.5% acrylamide. Second dimension SDS PAGE was performed using 8-18% T 16×18 cm polyacrylamide slab gels run in a Protean II XL multicell apparatus (Bio-Rad) at 4° C. Current conditions were 3 mA per gel for 6 hours followed by 15 mA per gel for 14 hours. Following second dimension electrophoresis proteins were fluorescently stained with SYPRO Ruby (Molecular Probes, Eugene, Oreg., USA) according to the manufacturer's instructions.

### (6) Image Analysis of 2D Gels

Gels were scanned using a Molecular Imager FX (Bio-Rad) equipped with a 488 nm external laser. Differential protein expression profiles were analysed using Z3 V 2.0 image analysis software (Compugen, Israel). Triplicate images from each of the preconditioning treatment (HS, CHX, and MK801) and control samples were used to compile a raw master reference gel composite. The composite gels generated from each group and pH gradient were then used to compare the protein profiles between control and preconditioning treatments. The acquired image analysis data was used to identify protein spots down-/up-regulated in preconditioning for subsequent identification by MADLI-TOF mass spectrometry. Changes greater than 1.7 fold in protein expression compared to control were considered significant. Differences in protein expression at the 1.7 fold level analysed by unpaired t-test, confirmed statistical significance at the 95% confidence limit.

### (7) Tryptic Digestion of Protein Spots

Protein spots were excised and placed in a 96 well microtitre plate for digestion. Gel pieces were washed three times in 50% v/v acetonitrile, 25 mM NH₄HCO₃, pH 7.8 and dried using a SpeedVac centrifuge. Protein in gel pieces was subject to tryptic digestion at 37° C. for 16 hours in 8 μl (0.014 μg/μL in 25 mM NH₄HCO₃, pH 7.8) sequencing grade trypsin (Promega, Madison, Wis., USA) solution. Peptides were extracted from the gel pieces using 8 μl of 10% (v/v) acetonitrile, 1% (v/v) trifluoroacetic acid solution then, desalted and concentrated using ZipTips (Millipore, Bedford, Mass., USA). A 1 μl aliquot was spotted onto a MALDI sample plate with 1 μl of matrix (α-cyano-hydroxycinnamic acid, 8 mg/mL in 50% v/v acetonitrile, 1% v/v TFA) and allowed to air dry.

### (8) Matrix Assisted Laser Desorption Ionisation-Time-of-Flight (MALDI-TOF) Mass Spectrometry

MALDI mass spectrometry was performed with a Micromass TofSpec 2E Time of Flight Mass Spectrometer. A nitrogen laser (337 nm) was used to irradiate the sample. Spectra were acquired in reflectron mode in the mass range 600 to 3500 Da. A near point calibration was applied and a mass tolerance of 50 ppm used. The peptide masses generated were used to search against Rodentia entries in SwissProt using ProteinProbe on MassLynx.

### Results

Overall CHX and MK801 preconditioning resulted in protein down-regulation, while HS resulted in the up-regulation of proteins. From the composite gel images, 158 of the most differentially expressed proteins were selected for protein identification by MADLI-TOF mass spectrometry.

Of the 158 protein spots selected, the protein or tentative protein(s) were identified in 94 cases, representing 51 different proteins (see FIG. 1). *Values for fold up-/down-regulation≧1.7 are statistically significant (p<0.5) and are highlighted in bold.

For four different closely related protein families (ACTB/ACTG, ARF1-3, HSC70/HSPA2, TUBA1-3/TUBA6), peptide masses generated from protein spots were not able to distinguish the specific protein. Different protein spots representing the same protein or closely related protein(s) occurred for 22 of the identified proteins and are likely to represent post-translational modifications or proteolytic fragments of the protein.

### Example 2

### Differential Protein Expression in EPO Preconditioned Neuronal Cells

### Materials and Methods

### (1) Cultivation of Cortical Neurons and EPO Preconditioning

Establishment of cortical cultures was as previously described in Example 1 and briefly outlined below.

Cortical tissue from E18-E19 rats was dissociated in Dulbecco's Modified Eagle Medium (DMEM; Invitrogen, Carlsbad, Calif., USA) supplemented with 1.3 mM L-cysteine, 0.9 mM NaHCO₃, 10 units/ml papain (Sigma, St. Louis, Mo., USA) and 50 units/ml DNase (Sigma) and washed in cold DMEM/10% horse serum. Neurons were resuspended in Neurobasal (NB; Life Technologies)/2% B27 supplement (Life Technologies), 1.6% fetal bovine serum (Life Technologies), 0.4% horse serum, 25 μM glutamate, 10 μM 2-mercaptoethanol, 12 μg/ml penicillin and 20 μg/ml streptomycin. The neuronal cell suspension was used to seed wells of a 6 well plate (9 cm²; Costar, USA), 35 mm glass dish or 96 well plated sized plastic/glass wells precoated with poly-D-lysine (40 μg/ml; 70-150K; Sigma). Six well plates and 35 mm glass dishes were seeded with 1.5 million neurons and the 96 well plate sized vessels with 50,000 neurons. Neuronal cultures were maintained in a CO₂ incubator (5% CO₂, 95% air balance, 98% humidity) at 37° C. On day in vitro (DIV) 4 one third of the culture medium was removed and replaced with fresh NB/2% B27 containing the mitotic inhibitor cytosine arabinofuranoside (final concentration 1 μM; Sigma), and on DIV 8 one half of the culture medium was replaced with NB/2% B27. At DIV 12 between 1-2% of cells in neuronal cultures stain positively for glial fibrillary acidic protein (GFAP). Erythropoietin preconditioning (EPO: 0.5 units/ml) consisted of adding EPO directly to neuronal cultures on DIV 11 or 12. EPO exposure was for 8, 12 or 24 hours before in vitro ischemia and for 12 hours before protein isolation for 2D electrophoresis. Controls consisted of DIV 12 untreated cortical neuronal cultures.

### (2) Recombinant Adenovirus Construction and Transfection of Neuronal Cultures

Recombinant adenovirus was used to up-regulate EPO expression in primary cortical neuronal cultures. The EPO expressing adenovirus was produced by first obtaining cDNA for the EPO protein by RT-PCR and cloning into pGEM. Sequence verified cDNA clones were then sub-cloned into a modified pShuttle vector (Stratagene) so that EPO cDNA expression was under the control of the rous sarcoma virus (RSV) promoter and the woodchuck post-transcriptional regulatory element (WE). The modified pShuttle vector also expressed green fluorescent protein (GFP) under the control of the CMV promoter. The modified pShuttle vector was then used to generate recombinant adenovirus expressing EPO and GFP(RSV:EPO-WE/CMV:GFP) using the AdEasy system (Stratagene). Control adenoviruses were also constructed and consisted of an adenovirus expressing red fluorescent protein (RFP) (RSV:RFP-WE/CMV:GFP), no gene (RSV:Empty-WE/CMV:GFP) and the anti-apoptotic gene Bcl-xl (RSV: Bcl-xl-WE/CMV:GFP). Recombinant adenoviruses were amplified in HEK 293 cells and purified using the BD Adeno-X virus purification kit (BD Biosciences Clontech, Calif., USA). Protein expression in recombinant adenoviruses was confirmed in transfected HEK 293 and cortical neuronal cultures by western analysis for RFP and Bclxl and ELISA for EPO (data not shown).

On DIV 9 neuronal culture wells (96 well plate format) were transfected with recombinant adenovirus by removing conditioned media from wells and adding 50 μl of fresh NB/2% B27 containing recombinant adenovirus at a multiplicity of infection (MOI) of 75 and 0.4% Booster 1 reagent (Gene Therapy Systems). In preliminary studies it was determined that transfection of neuronal cultures at 75 MOI produced a high degree of transgene expression (based on direct detection of RFP) with minimal toxicity. After 3 hours incubation adenovirus containing media was removed and replaced with 10 μl of a 50%/50% mix of conditioned and fresh NB/2% B27 media. Seventy two hours following adenovirus transfection neuronal cultures were subjected to in vitro ischaemia or cumene as described below.

### (3) In Vitro Ischaemia and Cumene Injury Models

In vitro ischaemia was performed in 96 well plate sized custom made glass wells. In this model, media from wells was removed, wells washed by adding and removing 315 μl balanced salt solution B (BSSB; mM: 116 NaCl, 5.4 KCl, 1.8 CaCl₂, 0.8 MgSO₄, 1 NaH₂PO₄; pH 7.0) and re-adding 50 μl of BSSB. Neuronal cultures were placed into an anaerobic chamber (Don Whitely Scientific, England) with an atmosphere of 5% CO₂, 10% H₂ and 85% argon, 98% humidity at 37° C. for 60 minutes. Following anaerobic incubation an equal volume of DMEM supplemented with 2% N2 was added before plating culture wells into a CO₂ incubator. Control cultures for both in vitro ischemia models received the same BSS wash procedures and media additions as ischaemic cultures, but were maintained in a CO₂ incubator.

Cumene induced oxidative stress was performed by removing media from 96 well plate neuronal culture wells and adding 100 μl DMEM/N2 medium containing cumene (20 mM). Cultures well were then incubated in a CO₂ incubator for 16-24 hours. Neuronal viability was measured and analysed as described below.

### (4) Assessment of Neuronal Viability and Statistical Analysis

Neuronal viability was assessed 24 hours after in vitro ischaemia qualitatively by nuclear morphology following staining with the fluorescent dye, propidium iodide and quantitatively by the MTS assay (Promega). The MTS viability assay measures the mitochondrial conversion of the tetrazolium salt to a water-soluble brown formazan salt, which is measured spectrophotometrically (495 nm). Although we did not distinguish between apoptotic and necrotic cell death following in vitro ischaemia, as indicated previously (Meloni 2001; Arthur et al., 2004) based on light microscope and nuclear staining the in vitro model results in predominantly apoptotic-like neuronal death. Neuronal viability in control cultures was treated as 100%. Viability data was analysed by ANOVA, followed by post-hoc Fisher's PLSD test. P<0.05% values were considered to be statistically significant.

### (5) Protein Isolation

See Section 4 in Example 1.

### (6) 2-Dimensional Gel Electrophoresis

See Section 5 in Example 1.

### (7) Image Analysis of 2D Gels

See Section 6 in Example 1.

### (8) Tryptic Digestion of Protein Spots

See Section 7 in Example 1.

### (9) Matrix Assisted Laser Desorption Ionisation-Time-of-Flight (MALDI-TOF) Mass Spectrometry

See Section 8 in Example 1.

### (10) Western Blotting

Total protein lysate (2 μg) was loaded into each lane of a 4-20% polyacrylamide gradient SDS gel (Invitrogen), electrophoresed and blotted onto PVDF membranes using protocols described for the NuPAGE electrophoresis system (Invitrogen). Membranes were incubated with primary antibody overnight at 4° C. followed by incubation with horseradish peroxidase labelled secondary antibody for 1 hour at room temperature. Secondary antibody was detected using the Enhanced Chemiluminescent (ECL) immunodetection system (Amersham). Antibodies used were; anti-Bcl-xl (SPA-760, Stressgen), anti-DsRed (BD Biosciences), and anti-β-tubulin (60181A, Pharmingen). To quantify the expression of each protein, autoradiographs were scanned into Adobe Photo-Proshop 5.0 and quantification of band intensity determined using NIH image 1.62 computer software. For each sample tubulin was used as a loading control and the expression of each protein normalised to tubulin protein levels.

### Results

### (1) EPO Preconditioning and 2D Gel Electrophoresis

Overall EPO preconditioning resulted in protein up-regulation. From the composite gel images, 84 of the most differentially expressed proteins were selected for protein identification by MADLI-TOF mass spectrometry, and the protein or tentative protein(s) were identified in 57 cases, representing 40 different proteins (See FIG. 2). Values for fold up-/down-regulation≧1.7 are statistically significant (p<0.5). A=Protein spot absent in treatment gel. N=Protein spot new in treatment gel.

Different protein spots representing the same protein or closely related protein(s) occurred for 13 of the identified proteins and are likely to represent post-translational modifications or proteolytic fragments of the protein. For three proteins (HSC70, STMN1, TPM5) different protein spots representing post-translational or proteolytic modifications of the same protein were observed to be up- and down-regulated.

### (2) EPO Adenovirus Transfection and Neuroprotection

Adenovirus mediated EPO overexpression protected neurons from cumene induced oxidative injury, by increasing neuronal survival from 5% to 45%.

### Example 3

### Differential Protein Expression in EPO Preconditioned Neuronal Cells

### Materials and Methods

### (1) Neuronal Cultivation

As per Examples 1 and 2.

### (2) Adenovirus Construction

Adenovirus vectors were used to upregulate specific proteins in primary cortical neuronal cultures. cDNA for proteins of interest was obtained by RT-PCR and cloned into pGEM. Sequence verified cDNA clones were then used to construct recombinant adenoviruses expressing genes of interest under the control of the rous sarcoma virus (RSV) promoter and the woodchuck post-transcriptional regulatory element (WPRE). The recombinant viruses also express the reporter GFP under the control of the CMV promoter. Protein of interest expression in recombinant adenoviruses was confirmed in transfected HEK and/or neuronal cultures. Control viruses consisted of an adenovirus expressing RFP, no gene (empty vector) and the anti-apoptotic gene Bcl-xl. Recombinant adenoviruses expressing the following genes have been constructed and several have been used in functional studies: Actin cytoplasmic 1 (ACTB), ATP synthase alpha chain (ATP5A), Elongation factor 1-alpha (EF1A1), Fatty acid-binding protein—brain (FABP7), Guanine nucleotide-binding protein G (O) (GNAO1), Protein kinase C zeta type (PKCZ), Phosphatidylethanolamine-binding protein (PEBP), Peroxiredoxin 2/Thioredoxin peroxidase 1 (PRDX2), Peptidyl-prolyl cis-transisomerase A/cyclophilin A (CyPA), Rho guanine dissociation inhibitor (GDI-1), SOD Cu/Zn (SOD1), Stathmin (STMN1), Voltage dependent anion channel/porin (VDAC1) and 14-3-3 protein gamma (YWHAG).

Of the adenoviruses constructed we have performed functional experiments using, PRDX2, CyPA, SOD1.

### (3) Adenovirus Transfection and CyPA Protein Incubation of Neuronal Cultures

On day in vitro (DIV) 9 neuronal culture wells (96 well plate format) were transfected with recombinant adenovirus by removing conditioned media from wells and adding 50 μl of fresh media (NB2^{o}) containing the desired dose of virus (MOI 25-100) and 0.4% Booster 1 reagent (Gene Therapy Systems). After 3 hours incubation adenovirus containing media was removed and replaced with 100 μl of a 50 μl/50 μl mix of conditioned and fresh media. Forty eight to 72 hours following adenovirus transfection neuronal cultures were subjected to in vitro ischaemia or cumene as described below.

For protein incubation, CyPA protein suspended in PBS was added to neuronal cultures (0.1-100 nM, final concentration) at the commencement of in vitro ischaemia or cumene exposure. The CyPA protein remained in neuronal cultures for the duration of the experiment.

### (4) In Vitro Ischaemia/Stroke Model (Transient Oxygen Glucose Deprivation)

To induce in vitro ischemia, culture medium in wells was first removed and 315 μl of glucose free balanced salt solution (BSS; mM: 116 NaCl, 5.4 KCl, 1.8 CaCl₂, 0.8 MgSO₄, 1 NaH₂PO₄; pH 7.3) added to each well. The 315 μl BSS was then removed and 50 μl of BSS re-added to the wells, before placing culture wells into an anaerobic chamber (Don Whitely Scientific, England) with an atmosphere of 5% CO₂, 10% H₂ and 85% argon, 98% humidity at 37° C. for 60 minutes. Reperfusion was performed by removing neuronal cultures wells from the anaerobic chamber, and immediately adding an equal volume (50 μl) of DMEM/N2 (containing 25 mM glucose, 0.5 mM glutamine, 26 mM NaHCO₃, 10 mM HEPES) supplemented with 2% N2 (Life Technologies) before incubation in a CO₂ incubator with an atmosphere of 5% CO₂, 95% air, 98% humidity at 37° C. Twenty four hours after in vitro ischaemia neuronal viability was measured using the MTS assay (Promega). Percentage neuronal survival in neuronal cultures treated with adenoviruses expressing genes of interest was compared to empty vector treated controls and data analysed with ANOVA followed by Fisher's test.

### (5) Cumene Oxidative Stress Injury Model

Cumene induced oxidative stress was performed by removing media from neuronal culture wells and adding 100 μl DMEM/N2 medium containing different concentrations of cumene (20-25 μM). Cultures well were then incubated in a CO₂ incubator for 16-24 hours. Neuronal viability was measured and analysed as described above.

### (6) PPIA Protein (Cyclophilin A=CyPA) and CyPA Receptor (CD147) Expression Following Ischaemic Preconditioning in the Rat Brain.

Rats were subjected to 3 minutes of preconditioning transient global ischaemia and at different time points post-ischaemia (6, 12, 24 48 hours) rats were sacrificed and hippocampal brain regions collected. Total protein was isolated from hippocampal tissue and CyPA protein expression analysed by western analysis.

### Results

Using recombinant adenovirus to overexpress different proteins in neuronal cultures prior to in vitro ischaemia and cumene we have found:
a) CyPA, PRDX2 and SOD1 improve neuronal survival following cumene exposure and CyPA improves neuronal survival following in vitro ischaemia;
b) adding CyPA protein (protein incubation) at the time of in vitro ischaemia and cumene insults is also neuroprotective; and
c) that the CyPA protein and CyPA receptor is up-regulated following ischaemic preconditioning in the rat brain.

The direct neuroprotective action of PPIA (cyclophilin A) protein on neuronal cultures indicates that the neuroprotective action of CyPA is transduced via its receptor (CD147).

### Example 4

### Neuroprotective Action of Cyclophilin A

### Materials and Methods

(1) Preparation of Shuttle Plasmid Encoding Rat CyPA cDNA.

Total rat brain RNA was purified from a male Sprague Dawley rat, reverse transcribed, and amplified by PCR using the oligonucleotides 5′-

containing a SalI restriction site (underlined) and a Kozak sequence (bold) and 5′-
containing a XhoI restriction site (underlined).

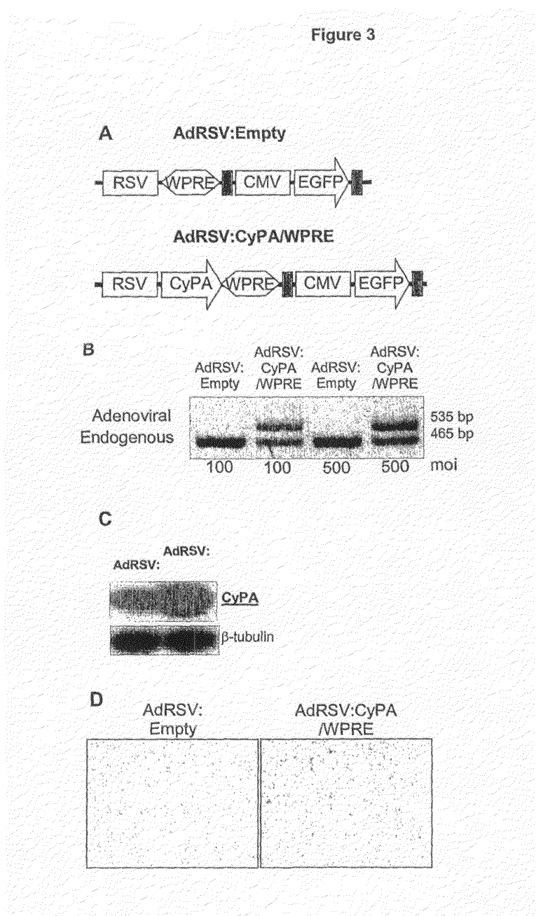

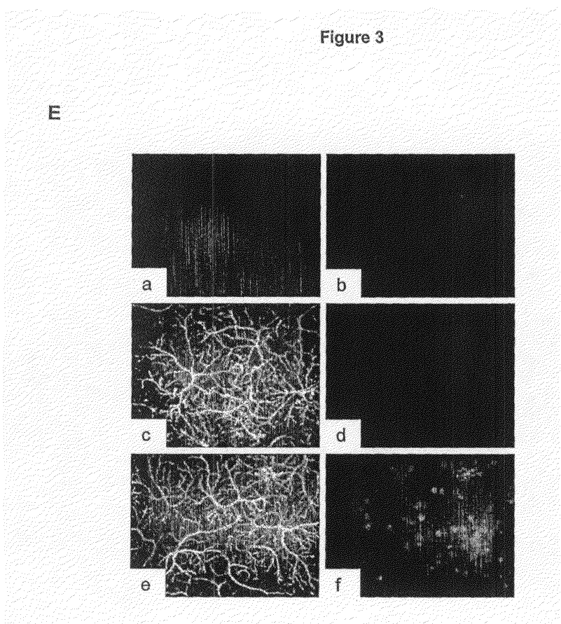

The resulting PCR product was gel purified then cloned into pGEM-Teasy (Promega, USA) for bi-directional sequence verification. Cyclophilin A cDNA was released by SalI and XhoI digestion, and directionally sub-cloned into our modified shuttle plasmid vector designated pRSV/WPRE, to create the shuttle plasmid pRSV:CyPA/WPRE (FIG. 3A).

### (2) Preparation of Recombinant Adenovirus

Recombinant adenovirus was prepared according to the method of He et al. (1998), with some modifications. Briefly, pRSV:CyPA/WPRE was linearised by Pmel digestion and introduced into E. coli strain BJ5183 carrying pAdeasy (Zeng et al. 2001), by electroporation (Gene Pulser II, Biorad). Recombinants were selected on media containing 50 μg/ml kanamycin, and their plasmid DNA checked by PacI digestion. HEK293 cells grown to 90% confluence in 25 cm² flasks were transfected with 3 μg of PacI linearised recombinant plasmid DNA using Lipofectamine-2000 (Invitrogen). Viral plaques appeared within 5-10 days and viral material used for subsequent amplification of the virus in HEK293 before purification and concentration using the Adeno-X virus purification kit (BD Biosciences). Infectious viral titres were determined by end-point dilution assay, as indicated by EGFP reporter expression.

### (3) Preparation of Cortical Neuronal Cultures

All animal procedures were approved by the University of Western Australia Animal Ethics Committee. Establishment of cortical cultures was previously described (Meloni et al. 2001), but briefly, cortical tissue from E18-E19 rats were dissociated in Dulbelcco's Modified Eagle Medium (DMEM; Invitrogen, USA) supplemented with 1.3 mM L-cysteine, 0.9 mM NaHCO₃ 10 units/ml papain (Sigma, USA) and 50 units/ml DNase (Sigma) and washed in cold DMEM/10% horse serum. Neurons were resuspended in Neurobasal (NB; Invitrogen) containing 2% B27 supplement (B27; Invitrogen). Before seeding, culture vessels, consisting of either 96 well sized plastic or glass wells (6 mm Dia.) were coated with poly-D-lysine (50 μg/mL; 70-150K; Sigma) and incubated overnight at room temperature (RT). The poly-D-lysine was removed and replaced with NB (containing 2% B27; 4% fetal bovine serum; 1% horse serum; 62.5 μM glutamate; 25 μM 2-mercaptoethanol; and 30 μg/mL streptomycin and 30 μg/mL penicillin). Neurons were plated to obtain around 10,000 viable neurons per well on day in vitro (DIV) 9.

Neuronal cultures were maintained in a CO₂ incubator (5% CO₂, 95% air balance, 98% humidity) at 37° C. On DIV 4 one third of the culture medium was removed and replaced with fresh NB/2% B27 containing the mitotic inhibitor, cytosine arabinofuranoside (CARA; Sigma) at 1 μM and on DIV 8 one half of the culture medium was replaced with NB/2% B27. On DIV 11, between 0.5-2% of cells in neuronal cultures stain positively for glial fibrillary acidic protein (Meloni et al. 2001). For astrocyte enriched neuronal cultures, CARA was omitted during cultivation.

### (4) Adenoviral Transfection

On DIV 9, the media was removed from cortical neuronal cultures and purified virus was diluted in 50 μl of NB/2% B27, to achieve the required multiplicity of infection (moi), and added to each well, and incubated for 3 h at 37° C. The virus containing media was removed and replaced by an equal mix of conditioned media and fresh NB/2% B27. Unless otherwise indicated, transfected neuronal cultures were used on DIV 12.

### (5) RT-PCR

On DIV 9, rat cortical neuronal cultures were transfected with either AdRSV:Empty or AdRSV:CyPA/WPRE at a moi of 100 and 500. On DIV 12, total RNA was extracted by the Trizol (Invitrogen) method and 100 ng was reverse transcribed using Oligo-dT primer (Promega, USA) and Retroscript (Ambion, USA). PCR products were derived by amplification under the following conditions; 25 cycles of (94° C.×30 s, 50° C.×30 s, 72° C.×45 s) using three sets of primers designed to differentiate between endogenous CyPA mRNA expression (465 bp band) and viral mediated CyPA mRNA expression (535 bp band). The oligonucleotides used were as follows; the common sense primer was 5′-tgggtcgcgtctgcttc-3′ (SEQ ID NO: 3; from the rat CyPA open reading frame) and the two anti-sense primers were 5′-aatgcccgcaagtcaaagaa-3′ (SEQ ID NO: 4; from the rat CyPA mRNA 3′ UTR) and 5′-gtaaaaggagcaacatag-3′ (SEQ ID NO: 5; from the 5′ end of the WPRE sequence).

(6) Semi-Quantitative Analysis of Rat Hippocampus CyPA mRNA

Total RNA was extracted from whole hippocampus of frozen brains using the Trizol method (Invitrogen). Following DNase treatment, 2 μg of total RNA was reverse transcribed using MMLV-RT and random decamers (Ambion) in a 20 μl reaction. For semi-quantitative PCR, CyPA was co-amplified with Universal 18S Internal Standards (Ambion). Primer sequences for CyPA are as follows; forward 5′-TGGGTCGCGTCTGCTTC-3′ (SEQ ID NO: 6) and reverse 5′-AATGCCGCGAAGTCAAAGAA-3′ (SEQ ID NO: 7). All reactions were performed using 2 μl of cDNA in a total volume of 50 μl. Forward and reverse primer concentrations were 200 ηM. Following a 3 min denaturation step at 94° C., PCR products were derived by amplification under the following conditions; 19 cycles of (94° C.×20 s, 57° C.×30 s, 72° C.×60 s). PCR products were electrophoresed in a 2% agarose gel and stained with SYBR Gold (Molecular Probes, USA). Gels were digitised using Kodak Digital Science (Eastman Kodak Co., USA) and quantified using NIH image.

### (7) Western Blotting

For protein extraction, brain tissue and cultured cells were lysed in buffer (50 mM Tris-HCl pH 7.5, 100 mM NaCl, 20 mM EDTA, 0.1% SDS, 0.2% deoxycholic acid, containing Complete™ protein inhibitor, Roche), vortexed briefly and clarified by centrifugation at 4° C. Protein concentrations were determined by the Bradford assay (Biorad, USA). Equivalent amounts of protein (5-10 μg per lane) were loaded and separated on 4-12% gradient SDS poly-acrylamide Bis-Tris mini-gels, (NuPAGE; Invitrogen) and transferred to a PVDF membrane. Membranes were blocked in PBS/T containing ovalbumin (1 mg/mL) for 1 min at room temperature before washing in PBS/T and PBS.

Membranes were incubated at 4° C. overnight in blocking solution containing primary antibody, washed and incubated in blocking solution containing HRP conjugated secondary antibody for 1 h at room temperature. Protein bands were detected using ECL Plus (Amersham, UK) and visualised by exposure to x-ray film (Hyperfilm; Amersham), scanned and quantified using NIH image. Primary antibodies used were; rabbit polyclonal anti-CyPA (1:25000; Biomol), mouse monoclonal anti-β-tubulin (0.5 μg/mL, Pharmingen, USA), mouse monoclonal anti-phospho ERK1/2 (1:5000; Santa Cruz), rabbit polyclonal anti-ERK1/2 (1:10000; Santa Cruz) and goat polyclonal anti-CD147 (1:10000; Santa Cruz). Secondary antibodies were donkey anti-rabbit IgG (1:25,000-1:50,000; Amersham), sheep anti-mouse IgG (1:10,000-1:20,000; Amersham) and rabbit anti-goat IgG (Zymed).

### (8) Imaging

Bright field and fluorescence imaging: Image acquisition was performed using an Olympus IX70 fluorescent microscope fitted with a cooled CCD digital camera (DP70, Olympus) under software control (DP controller, Olympus).

### (9) Immunocytochemistry

Neuronal cultures were fixed with formalin (4%, in PBS; pH 7.5) for 1 h, washed 3 times in PBS treated with hydrogen peroxide (3%, in PBS for 5-10 min) and washed in PBS Tween 20 (0.1%). After blocking with horse serum (20 min), cultures were incubated with primary antibody overnight at 4° C., washed in PBS Tween 20 and probed with a biotin conjugated secondary antibody (DAKO).

Immunoreactivity was detected using horseradish peroxidase conjugated to strepavidin (DAKO) and DAB substrate (SigmaFast). Primary antibodies used were; rabbit polyclonal anti-CyPA (1:1000; Biomol), mouse monoclonal anti-GFAP (mouse IgG1 isotype) derived from clone G-A-% (Sigma), goat polyclonal anti-CD147 (1:500; Santa Cruz) and rabbit polyclonal neuron specific enolase (DAKO kit). For immunofluoresence detection, secondary antibodies used were goat polyclonal anti-IgG Alexafluor 546 (1:100; Molecular Probes) and goat anti-mouse IgG Alexafluor 488 (1:100; Molecular Probes).

### (10) Cell Death Assays

### (a) In Vitro Ischemia

Prior to in vitro ischemia, cultures were treated with recombinant human cyclophilin A (rhCyPA, Biomol), for 15 min at 37° C. Exposure of neuronal cultures to in vitro ischemia was performed by removing media from each well, washing in 315 μL balanced salt solution (BSS; mM: 116 NaCl, 5.4 KCL, 1.8 CaCl₂, 0.8 MgSO₄, 1 NaH₂PO₄; pH 7.0) and re-adding 50 μL of BSS. A parallel set of normal cultures or cultures transfected with the control vector, AdRSV:Empty, received glutamate receptor antagonists 1 μM MK801/10 μM 6-cyano-7-nitroquinoxaline (CNQX, Tocris, USA).

Neuronal cultures were placed into an anaerobic chamber (Don Whitely Scientific, England) with an atmosphere of 5% CO₂, 10% H₂ and 85% argon, 98% humidity at 37° C. for 50 min. Following anaerobic incubation an equal volume of DMEM containing 2% N2 supplement (Invitrogen) was added to each well before placing wells into a CO₂ incubator at 37° C. Control cultures received the same BSS wash procedures and media additions as ischemic cultures, but were maintained in a CO₂ incubator.

Neuronal viability was assessed 24 h after in vitro ischemia using the MTS assay (Promega). Although we did not distinguish between apoptotic and necrotic cell death following in vitro ischemia, as reported previously (Meloni et al 2001; Arthur et al. 2004), based on light microscopy and nuclear staining, this model results in predominantly apoptotic-like neuronal death.

### (b) Cumene Hydroperoxide (Cumene) Treatment

The culture media from normal, or adenoviral transfected neuronal cultures was removed and replaced with 100 μl of DMEM/N2 1% containing freshly prepared cumene (Sigma) at the required concentration. A parallel set of normal cultures or cultures transfected with the control vector, AdRSV:Empty were given the glutamate receptor antagonists 1 μM MK801/10 μM 6-cyano-7-nitroquinoxaline (CNQX, Tocris, USA). For CyPA treated cultures, recombinant human rhCyPA (Biomol), was added with cumene. Cumene was diluted in ethanol as a 100× stock. Cell survival was assessed 24 hours later using the MTS assay.

### (c) Statistics

Neuronal viability in control cultures was treated as 100%. Viability data was analysed by ANOVA, followed by post-hoc Fischer's PLSD test. P values<0.05% were considered statistically significant.

### Results

### (1) Construction of Recombinant Adenovirus to Over-Express CyPA in Cortical Neuronal Cultures

The expression cassettes for the control adenovirus and adenovirus used to over-express CyPA is presented schematically in FIG. 3A. Successful adenoviral transfection of neuronal cultures was confirmed by EGFP reporter expression (data not shown). Subsequently, adenoviral mediated CyPA over-expression in cortical neuronal cultures was confirmed by RT-PCR (FIG. 3B) and Western analysis (FIG. 3C).

Immunocytochemistry of cultures transfected with AdRSV:CyPA/WPRE showed variable, but increased CyPA staining in neurons compared with neurons in cultures transfected with AdRSV:Empty (FIG. 3D). The variable CyPA staining is likely to reflect variability in the number of viral particles infecting neurons, as EGFP reporter expression (not shown) correlated with CyPA staining intensity. Using double immunofluorescence (summarised in FIG. 3E) of astrocyte enriched cultures, we detected CyPA staining in neurons, but not astrocytes.

### (2) Adenovirus Mediated CyPA Over-Expression Attenuates Neuronal Death Caused by Oxidative Stress and In Vitro Ischemia

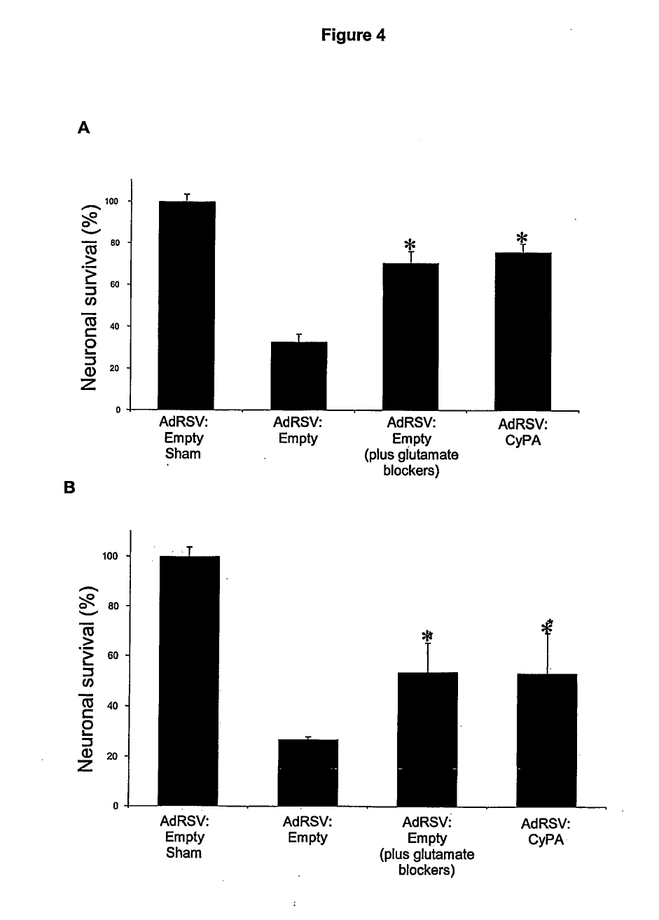

In a dose response experiment, we found exposure of cortical neuronal cultures to 25 μM cumene reduced cell survival to 15-30% (data not shown), a level comparable to that reported for PC12 cells (Vimard et al. 1996). Adenoviral mediated CyPA over-expression significantly increased neuronal survival following cumene exposure from 33% to 76% (FIG. 4A). In addition, adenoviral mediated CyPA over-expression increased neuronal survival following in vitro ischemia from 27% to 53% (FIG. 4B). Glutamate receptor antagonists, used as positive controls in both the cumene and in vitro ischemia models increased neuronal survival to 70% and 54% respectively.

(3) Cyclophilin A mRNA, But not Protein, is Increased in the Rat Hippocampus Following Preconditioning Ischemia

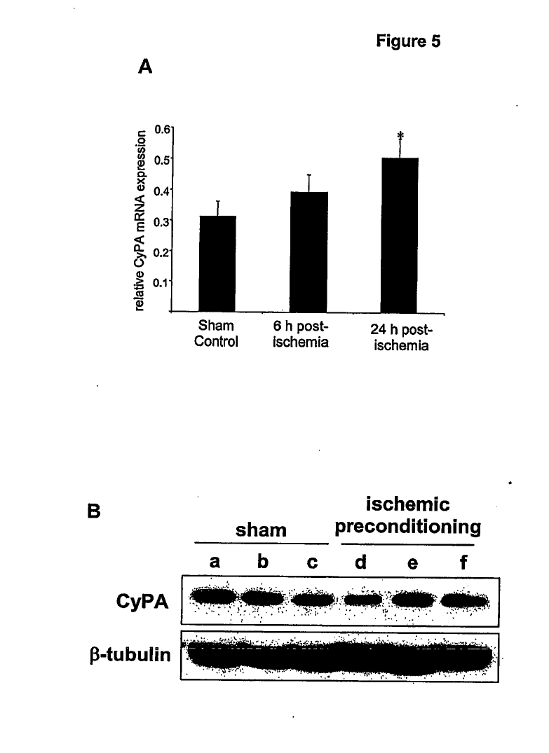

Using semi-quantitative RT-PCR analysis we observed a statistically significant increase in CyPA mRNA expression in the rat hippocampus at 24 h post preconditioning ischemia compared with a control group of animals (FIG. 5A). Western analysis of total hippocampus protein lysates did not show any increase in CyPA expression at 24 h following 3 min of preconditioning ischemia (FIG. 5B)

### (4) Neuronal Cultures Express CD147

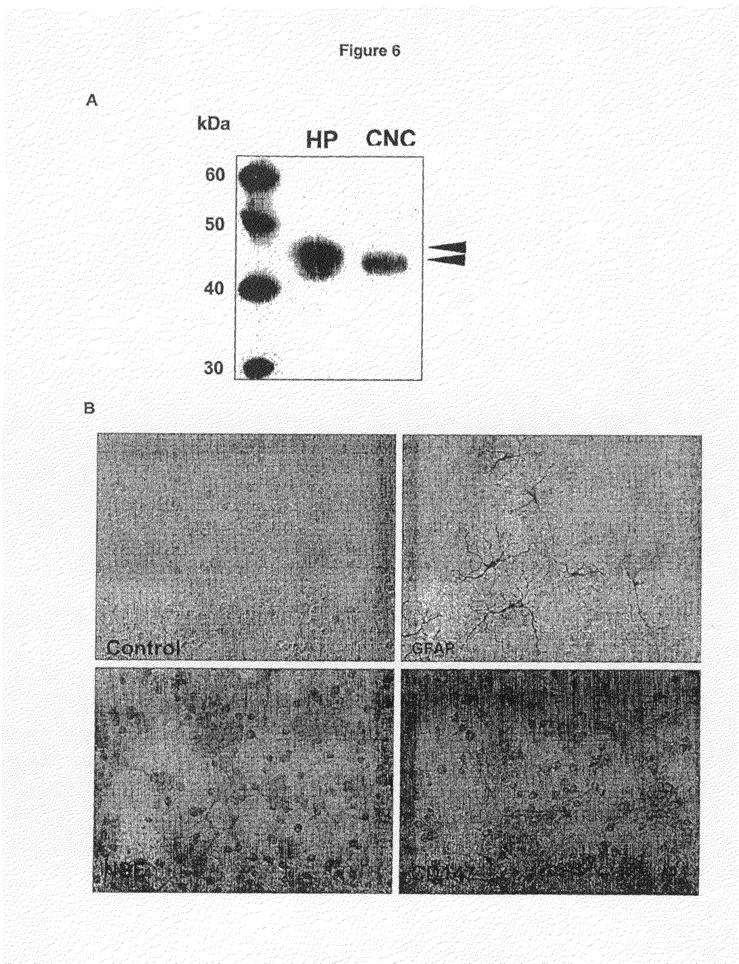

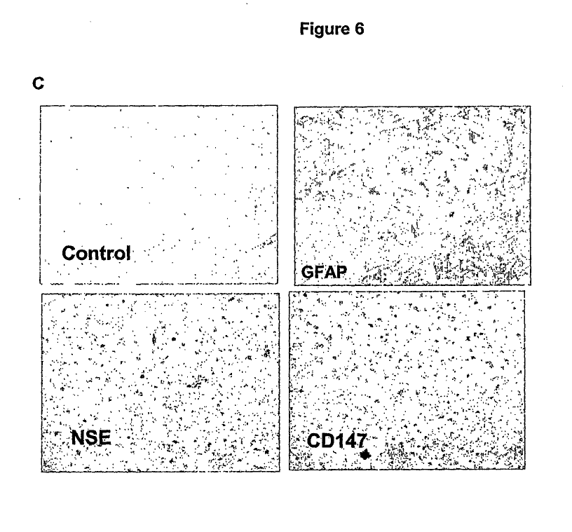

Using Western analysis, we detected CD147 immunoreactive protein in lysates prepared from total rat hippocampi and cortical neuronal cultures (FIG. 6A). Both protein species were of a similar molecular weight, and fell within the reported range 43-66 kDa for this receptor (Muramatsu and Miyauchi, 2003).

Immunocytochemistry revealed strong staining for the CD147 receptor in neuronal cultures (FIG. 6B). The staining pattern observed for CD147 closely correlated with the staining pattern observed for the neuronal marker, neuron specific enolase (NSE; FIG. 6B). We did not detect any cells resembling astrocytes staining for the CD147, despite 1-2% of cells in the neuronal cultures staining positively for the astrocytic marker GFAP (FIG. 6B). Immunocytochemistry for CD147 in neuronal cultures enriched for astrocytes also failed to reveal clear CD147 staining in astrocytes, while positive staining was still obtained in neurons (FIG. 6C).

### (5) Exogenous CyPA Activates ERK1/2 in Neuronal Cultures.

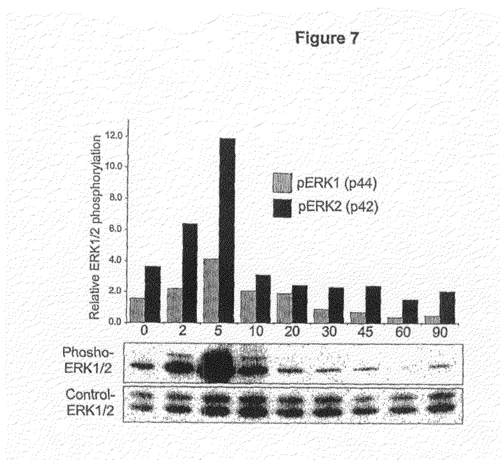

To determine if exogenously applied CyPA can mediate ERK1/2 activation we exposed neuronal cultures to rhCyPA protein (100 nM) and the results are summarised in FIG. 7. Addition of rhCyPA induced a rapid phosphorylation of ERK1 and ERK2, which peaked at 5 min, before returning to basal levels. At 5 min ERK1 (p44) activation increased 2.6 fold and ERK2 (p42) 3.3 fold.

### (6) Exogenous CyPA Attenuates Neuronal Death Caused by Oxidative Stress and in Vitro Ischemia

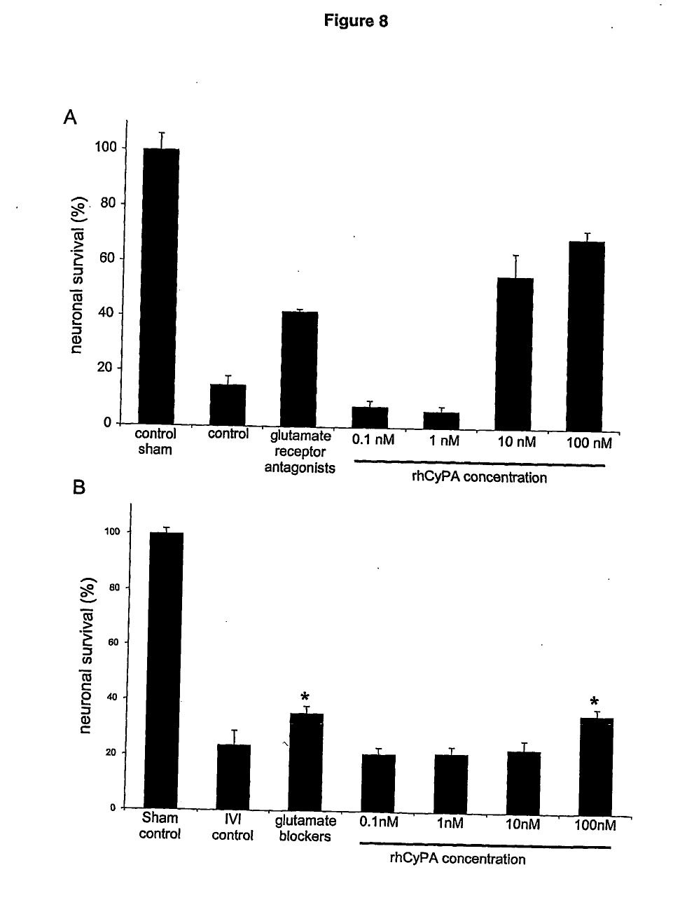

Exogenous application of rhCyPA to neuronal cultures prior to the commencement of cumene exposure and in vitro ischemia significantly increased neuronal survival (FIGS. 8A and 8B). Following cumene exposure, CyPA doses of 10 nM and 100 nM increased neuronal survival from 15% to 56% and 70% respectively. Following in vitro ischemia, a rhCyPA dose of 100 nM increased neuronal survival from 24% to 35%. Glutamate receptor antagonists increased neuronal survival to 42% and 35% following exposure to cumene and in vitro ischemia respectively.

### REFERENCES

1. Arthur P. G., Lim S. C., Meloni B. P., Munns S. E., Chan A. and Knuckey N. W. (2004) The protective effect of hypoxic preconditioning on cortical neuronal cultures is associated with increases in the activity of several antioxidant enzymes. Brain Res 1017, 146-154.
2. He T. C., Zhou S., da Costa L. T., Yu J., Kinzler K. W. and Vogelstein B. (1998) A simplified system for generating recombinant adenoviruses. Proc Natl Acad Sci USA 95, 2509-2514.
3. Meloni B. P., Majda B. T. and Knuckey N. W. (2001) Establishment of neuronal in vitro models of ischemia in 96-well microtiter strip-plates that result in acute, progressive and delayed neuronal death. Neuroscience 108, 17-26.
4. Meloni B. P., Majda B. T. and Knuckey N. W. (2002) Evaluation of preconditioning treatments to protect near-pure cortical neuronal cultures from in vitro ischemia induced acute and delayed neuronal death. Brain Res 928, 69-75.
5. Muramatsu T. and Miyauchi T. (2003) Basigin (CD147): a multifunctional transmembrane protein involved in reproduction, neural function, inflammation and tumor invasion. Histol Histopathol 18, 981-987.

The claims defining the invention are as follows:

1. A method of controlling neurodegeneration by increasing CD147 receptor signalling on neurons.

2. A method according to claim 1 wherein CD147 receptor signalling is increased by increasing the expression of CD147 on neurons.

3. A method according to claim 1 wherein CD147 receptor signalling is increased by increasing signalling efficiency.

4. A method according to claim 2 wherein expression of CD147 on neurons is increased using a DNA based therapy

5. A method according to claim 4 wherein DNA encoding CD147 is introduced into neurons to result in an increase in CD147 expression relative to non-treated cells.

6. A method according to claim 5 wherein the introduced DNA is adapted to be transcribed at high levels.

7. A method according to claim 5 or 6 wherein the introduced DNA encodes a modified CD147 that has enhanced ligand binding affinity.

8. A method according to claim 2 wherein CD147 expression is increased through the use of an agent that (i) increases transcription of the CD147 DNA into mRNA and/or (ii) increases the translation of mRNA coding for CD147.

9. A method according to claim 1 wherein CD147 receptor signalling is increased through the use of a ligand adapted to bind CD147 and evoke receptor signalling.

10. A method for screening a compound for neuroactivity comprising contacting a candidate with CD147 and assessing binding and or receptor signaling.

11. A method according to claim 10 comprising the steps of: (i) preparing a reaction mixture of the CD147 and the candidate compound under conditions and for a time sufficient to allow the two components to interact and bind, thus forming a complex; and (ii) detecting the complex.

12. A method according to claim 10 and 11 wherein CD147 or a fusion protein thereof or the candidate is attached to a solid phase.

13. A method according to claim 12 wherein the solid phase is a microtiter plate.

14. A method according to any one of claims 10 to 13 wherein at least one of the CD 147 and the candidate are cell bound.

15. A screening method comprising the steps of: (i) detecting the presence and/or measuring the level at least one of CD147, CyPA or a functional variant thereof in a patient; and (ii) comparing the result from (i) with the reference measure indicate of normality.

16. A method for controlling neural degeneration comprising the step of contacting a neuron with an effective amount of CyPA or a functional equivalent thereof.

17. A method according to claim 16 wherein the control of neural degeneration comprising complete removal of neural degeneration.

18. A method for treating a disease or disorder associated with neural degeneration comprising the step of administering to a subject an effective amount of CyPA or a functional equivalent thereof.

19. A method according to claim 18 wherein the disease or disorder is selected from the group consisting of: conditions characterized by cerebral ischemia, such as stroke; and other conditions characterized by progressive neuronal degeneration, such a Alzheimer's Disease, Parkinson's Disease, Motor Neuron Disease and any neurodegeneration and neuronal loss due to trauma and spinal cord damage.

20. Use of CyPA or a functional variant thereof as a prophylactic to reduce or prevent neuronal degeneration.

21. Use according to claim 20 wherein the disease or disorder is selected from the group consisting of: conditions characterized by cerebral ischemia, such as stroke; and other conditions characterized by progressive neuronal degeneration, such as Alzheimer's disease, Parkinson's Disease, Motor Neurone Disease and any neurodegeneration and neuronal loss due to trauma and spinal cord damage.

22. A method for reducing the degeneration of neurons comprising the step of contacting the neurons with an effective amount of CyPA or a functional equivalent thereof.

23. A method according to claim 22 wherein the neurons are CA1 hippocampal neurons.

24. A method according to any one of claims 16 to 19 or 22 to 23 or a use according to any one of claims 20 or 21 wherein the CyPA or functional variant thereof is delivered by implanting certain cells that have been genetically engineered to express and secrete CyPA or a functional variant thereof.

25. A method according to any one of claims 16 to 19 or 22 to 23 or a sue according to any one of claims 20 or 21 wherein the CyPA or functional variant thereof is delivered by implanting a gene therapy construct encoding CyPA or a functional variant thereof operably linked to a constitutive or inducible promoter.

26. A pharmaceutical or veterinary composition comprising CyPA or a functional variant thereof and a pharmaceutically acceptable carrier.


SEQUENCE LISTING (7 sequences)

SEQ ID NO: 1 - DNA, 36 bp, Artificial
  Synthetic oligonucleotide
gtcgacccaccatggtcaaccccaccgtgttcttcc

SEQ ID NO: 2 - DNA, 36 bp, Artificial
  Synthetic oligonucleotide
ctcgagttagagttgtccacagtcggagatggtgat

SEQ ID NO: 3 - DNA, 17 bp, Artificial
  Synthetic oligonucleotide
tgggtcgcgtctgcttc

SEQ ID NO: 4 - DNA, 20 bp, Artificial
  Synthetic oligonucleotide
aatgcccgcaagtcaaagaa

SEQ ID NO: 5 - DNA, 18 bp, Artificial
  Synthetic oligonucleotide
gtaaaaggagcaacatag

SEQ ID NO: 6 - DNA, 17 bp, Artificial
  Synthetic oligonucleotide
tgggtcgcgtctgcttc

SEQ ID NO: 7 - DNA, 20 bp, Artificial
  Synthetic oligonucleotide
aatgccgcgaagtcaaagaa